SEQUENCE LISTING (34 sequences)

SEQ ID NO: 1 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 2 - protein, 16 aa, Artificial sequence
  Synthetic peptide
IRTPKISKPIKFELSG

SEQ ID NO: 3 - protein, 16 aa, Mus musculus
IRTPKIAKPVKFELSG

SEQ ID NO: 4 - protein, 16 aa, Rattus norvegicus
IRTPKIAKPVKFELSG

SEQ ID NO: 5 - protein, 16 aa, Bos taurus
IRTPKISKPIKFQLSG

SEQ ID NO: 6 - protein, 16 aa, Sus scrofa
IRTPKISKPVKFELSG

SEQ ID NO: 7 - protein, 16 aa, Notophthalmus viridescens
IRTPKISKPVKFELSG

SEQ ID NO: 8 - protein, 16 aa, Xenopus laevis
IRTPKISKPVKFEFSG

SEQ ID NO: 9 - protein, 13 aa, Homo sapiens
PKISKPIKFELSG

SEQ ID NO: 10 - protein, 16 aa, Artificial sequence
  Synthetic peptide
AAAAKISKPIKFEKSG

SEQ ID NO: 11 - protein, 17 aa, Artificial sequence
  Synthetic peptide
IRTPAAAAAPIKFELSG

SEQ ID NO: 12 - protein, 16 aa, Artificial sequence
  Synthetic peptide
IRTPKISKAAAAELSG

SEQ ID NO: 13 - protein, 16 aa, Artificial sequence
  Synthetic peptide
IRTPKISKPILFAAAA

SEQ ID NO: 14 - DNA, 26 bp, Artificial sequence
  Synthetic primer
atcgggacatctcccgatcccctatg

SEQ ID NO: 15 - DNA, 59 bp, Artificial sequence
  Synthetic primer
agtgcgccgctgctgccaaaatctccaagcctatcaagtttgagctttctggctgcacc

SEQ ID NO: 16 - DNA, 39 bp, Artificial sequence
  Sytnthetic primer
gcatccgtactcccgcgcgccgcgcctatcaagtttgag

SEQ ID NO: 17 - DNA, 39 bp, Artificial sequence
  Synthetic primer
cccaaaatctccaaggctgccgcggctgagctttctggc

SEQ ID NO: 18 - DNA, 37 bp, Artificial sequence
  Synthetic primer
gcctatcaagtttgcggctgctgcctgcaccagcatg

SEQ ID NO: 19 - protein, 7 aa, Artificial sequence
  Synthetic peptide
GVCTDGR

SEQ ID NO: 20 - protein, 7 aa, Mus musculus
GVCTDGR

SEQ ID NO: 21 - protein, 7 aa, Rattus norvegicus
GVCTDGR

SEQ ID NO: 22 - protein, 7 aa, Bos taurus
GVCTDGR

SEQ ID NO: 23 - protein, 7 aa, Sus scrofa
GVCTDGR

SEQ ID NO: 24 - protein, 7 aa, Notophthalmus viridescens
GVCTDGR

SEQ ID NO: 25 - protein, 7 aa, Xenopus laevis
GVCTDGR

SEQ ID NO: 26 - protein, 14 aa, Artificial sequence
  Synthetic peptide
EENIKKGKKCIRPT

SEQ ID NO: 27 - protein, 17 aa, Artificial sequence
  Synthetic peptide
TPKISKPIKFELSGCTS

SEQ ID NO: 28 - protein, 10 aa, Artificial sequence
  Synthetic peptide
TSMKTYRAKF

SEQ ID NO: 29 - protein, 14 aa, Artificial sequence
  Synthetic peptide
CTPHRTTTLPVEFK

SEQ ID NO: 30 - protein, 18 aa, Artificial sequence
  Synthetic peptide
FKCPDGEVMKKNMMFIKT

SEQ ID NO: 31 - protein, 7 aa, Artificial sequence
  Synthetic peptide
MFIKTCA

SEQ ID NO: 32 - protein, 5 aa, Artificial sequence
  Synthetic peptide
ACHYN

SEQ ID NO: 33 - protein, 12 aa, Artificial sequence
  Synthetic peptide
CPGDNDIFESLY

SEQ ID NO: 34 - protein, 11 aa, Artificial sequence
  Synthetic peptide
LYYRKMYGDMA

(19) United States
(12) Patent Application Publication
(10) Pub. No.: US 2005/0153395 A1
(43) Pub. Date: Jul. 14, 2005

(54) Unique integrin binding site in connective tissue growth factor (CTGF)

(75) Inventors: David R. Brigstock, Dublin, OH (US); Runping Gao, Columbus, OH (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: CHILDREN'S HOSPITAL INC., Columbus, OH (US)

(21) Appl. No.: 10/955,860

(22) Filed: Sep. 29, 2004

Related U.S. Application Data
(60) Provisional application No. 60/506,891, filed on Sep. 29, 2003.

(51) Int. Cl. C07K 14/74; C07K 16/28; A61K 38/17; C07H 21/04

(52) U.S. Cl. 435/69.1; 435/320.1; 435/325; 530/350; 536/23.5; 514/12; 530/388.22

(57) ABSTRACT

The invention relates to a connective tissue growth factor peptide that encompasses a CTGF binding site for an integrin such as αᵥβ₃ or α₅β₁ and uses therefor. The invention also relates to agonists and inhibitors of the CTGF-integrin binding and methods of treating and preventing CTGF-related disorders.

### RELATED APPLICATIONS

The present application claims priority benefit from U.S. Provisional Application 60/506,901 filed Sep. 29, 2003, which is incorporated herein by reference in its entirety.

### FIELD OF INVENTION

The invention relates to connective tissue growth factor peptides that encompasses a binding site for an integrin and uses therefor. The invention also relates to inhibitors of CTGF and integrin binding and methods of treating and preventing CTGF-related disorders.

### BACKGROUND

Connective tissue growth factor (CTGF) has emerged as one of six new genes (the others are CYR61, NOV, WISP-1, -2, and -3) that have been classified into a group of structurally related molecules termed the “CTGF/CYR61/NOV” (“CCN”) family (Brigstock, Endocr Rev; 20, 189-206, 1999). Connective tissue growth factor (CTGF) is a growth and chemotactic factor for fibroblasts, myofibroblasts, epithelial cells, smooth muscle cells, neural cells and endothelial cells that is involved in critical biological processes including embryogenesis, placentation, wound healing, angiogenesis, and fibrosis. The CTGF primary translation product is a 349 amino acid protein that contains a 26-residue signal peptide. After signal peptide cleavage, the secreted form of CTGF (˜38kDa) contains 38 cysteine residues which are predicted to form four evolutionarily conserved structural modules. Module 1 is an insulin-like growth factor (IGF) binding domain, module 2 is a von Willebrand Type C (VWC) domain, module 3 is a thrombospondin-1 (TSP-1) domain, and module 4 is a C-terminal (CT) domain that may contain a cystine knot. The full length CTGF protein (“CTGF₁₋₄”) is susceptible to limited proteolysis yielding C-terminal fragments comprising essentially module 4 alone (“CTGF₄”) or modules 3 and 4 (“CTGF₃₋₄”). These CTGF isoforms are present in vivo, highly stable, bind to heparin and promote DNA synthesis, promote transdifferentiation of epithelial cells into myofibroblasts, production of alpha smooth muscle actin (αSMA), stimulate fibrosis in vivo, and promote cell adhesion. Sequences that are similar to CTGF₄, the C-terminal module of CTGF, also occur in the C termini of a variety of unrelated extracellular mosaic proteins (Bork, FEBS Lett; 327, 125-130 (1993). Six of the 10 cysteine residues in the CTGF₄ appear to adopt the cysteine knot motif found in TGF-β1, NGF, and PDGF.

CTGF is known to be a TGF-β1-induced immediate early gene (Brunner et al. DNA Cell Biol; 10: 293-300, 1991; Igarashi et al. Mol Biol Cell; 4: 637-45, 1993). TGF-β and CTGF share pro-fibrogenic properties whereas anti-inflammatory and immunosuppressive properties are unique to TGF-β. TGF-β-induced collagen production is antagonized by CTGF antibodies or antisense oligonucleotides in normal rat kidney cells (NRK) and human fibroblasts (Duncan et al., Faseb J; 13: 1774-86, 1999). TGF-β is able to support anchorage-independent growth of NRK cells, a process that is antagonized by CTGF antibodies or antisense oligonucleotides (Kothapalli et al., Cell Growth Differ; 8: 61-8, 1997). Additionally, subcutaneous injection of TGF-β into neonatal mice, which causes a rapid increase in the amount of granulation tissue comprising connective tissue cells and abundant ECM, results in enhanced levels' of CTGF mRNA in connective tissue fibroblasts. Finally, injection of CTGF causes a very similar fibrotic reaction as TGF-β and is not mimicked by other growth factors (Shinozkai et al., Biochem Biophys Res Commun; 240: 292-7, 1997, Frazier et al., J Invest Dermatol; 107: 404-11, 1996; Mori et al., J Cell Physiol 181: 153-9, 1999; Ball et al., Reproduction; 125: 271-84, 2003).

Integrins are cell surface receptors that are composed of two subunits, α and β. Each αβ combination has its own binding specificity and signaling properties. Integrins are involved in cell-matrix and cell-cell interactions (Giancotti et al., Science 285, 1028-1033, 1999; Hynes, Cell 110, 673-687, 2002). Conventional growth factors and integrins are known to interact. For example, αᵥβ₃ interacts with insulin, PDGF, and VEGF receptors (Schneller et al., EMBO J 16, 5600-5607, 1997; Soldi et al., Embo J 18, 882-892, 1999) and mediates endothelial cell adherence to FGF-2 (Rusnati et al., Mol. Cell Biol.; 8:2449-2461). Similarly, integrin α₅β₁ interacts with IGF-1 and VEGF. (Kabir-Salmani et al., Mol. Hum. Reprod. 10(2): 91-97, 2004; Wijelath et al., J. Vasc. Surg., 39(3): 655-660).

The full length Cyr61, NOV, and mouse CTGF promote human endothelial cell adhesion through αᵥβ₃ (Babic et al., Mol Cell Biol 19, 2958-2966, 1999) and Cyr61 promotes human skin fibroblasts adhesion through α₆β₁ and heparin sulfate proteoglycans (HSPG) (Chen et al., J Biol Chem 275, 24953-24961, 2000; Chen et al., J Biol Chem 276, 10443-10452, 2001). Integrin αᵥβ₃ binds to a broad repertoire of RGD-containing ligands including vitronectin, fibronectin, fibrinogen, von Willebrand factor, and thromspondin (Cheresh, Proc Natl Acad Sci USA 84, 6471-6475, 1987; Smith et al., J Biol Chem 269, 960-967, 1994). In addition, various non-RGD ligands of αᵥβ₃ have also been recognized, such as CD31/PECAM-1 (Piali et al., J Cell Biol 130, 451-460, 1995), matrix metalloproteinase (MMP-2) (Brooks et al., Cell 85, 683-693, 1996), CTGF (Leu et al. J Biol Chem 277, 46248-46255, 2002), NOV (Babic et al., Mol Cell Biol 19, 2958-2966, 1999), and FGF-2 (Rusnati et al., Mol Biol Cell 8, 2449-2461, 1997). Integrin α₅β₁ is involved in mediating adhesion of fibroblasts or pancreatic stellate cells to CTGF and of endothelial cells to NOV (Chen. et al., Mol Biol. Sci. epub September 2004; Lin et al., 278(26): 24200-8, 2003; Ellis et al., J. Vasc. Res. 40(3): 234-43, 2003) Integrin α₅β₁ is also a receptor for well-characterized cell adhesion molecules such as fibronectin, plasminogen, and fibrillin-1 (Takagi et al., EMBO J 22(18): 4607-15, 2003; Lishko et al., Bood 104(3): 719-26, 2004; Bax et al., J. Biol. Chem. 278: 34605-16, 2003).

As CTGF-integrin binding is known to play a role in various biological activities. These activities are involved in CTGF related disorders such as fibroproliferative disorders, connective tissue disorders, hyperproliferative disorders and angiogenesis-related disorders. Thus, there is a need to identify modulators of the interaction between CTGF and integrins, such as integrin αᵥβ₃ or integrin α₅β₁.

### SUMMARY OF INVENTION

The present invention provides for a peptide comprising residues 257-272 of human CTGF (SEQ ID NO: 1). This peptide is denoted herein as “CTGF[257-272].” This peptide encompasses the binding site for integrin αᵥβ₃. The sequence of this peptide is IRTPKISKPIKFELSG (SEQ ID NO: 2).

The present invention provides for a peptide comprising residues 285-291 of human CTGF (SEQ ID NO: 1). This peptide is denoted herein as “CTGF[285-291].” This peptide encompasses the binding site for integrin α₅β₁. The sequence of this peptide is GVCTDGR (SEQ ID NO: 19).

The invention provides for isolated peptides comprising a fragment of the amino acid sequence of SEQ ID NO: 1 that binds to an integrin such as αᵥβ₃ or α₅β₁. For example, the peptides of the invention include isolated peptides comprising the amino acid sequence of SEQ ID NO: 2, isolated peptides comprising residues 257-272 of SEQ ID NO: 1, isolated peptides comprising the amino acid sequence of SEQ ID NO: 19 and isolated peptides comprising residues 285-291 of SEQ ID NO: 1.

The invention also provides for the peptides corresponding to integrin binding sites in CTGF homologs such as murine CTGF (SEQ ID NO: 3; residues 256-271 or SEQ ID NO: 20; residues 285-291 of Genbank Accession No. NP_{—}034347), rat CTGF (SEQ ID NO: 4; residues 255-270 or SEQ ID NO: 21; residues 285-291 of Genbank Accession No. NP_{—}071602), bovine CTGF(SEQ ID NO: 5; residues 257-272 or SEQ ID NO: 22; residues 285-291 of Genbank Accession No. NP_{—}776455), pig CTGF (SEQ ID NO: 6; residues 257-272 or SEQ ID NO: 23 residues 285-291 of Genbank Accession No. AAD0074), Notophthalmus viridescens CTGF (SEQ ID NO: 7, residues 255-270 or SEQ ID NO: 24; residues 285-291 of Genbank Accession No. CAB65965), and Xenopus laevis CTGF (SEQ ID NO: 8; residues 251-266 or SEQ ID NO: 25; residues 285-291 of Genbank Accession No. AAB67639). The presence of the amino acid sequence of SEQ ID NO: 2 and SEQ ID NO: 19 may be found in CTGF homologues and other proteins. These integrin binding sites may be present in other CCN family members or any other integrin binding protein.

The invention also provides for a polynucleotide sequence encoding a peptide of the invention. Particularly, the invention provides for a polynucleotide sequence that encodes a peptide comprising a fragment of the amino acid sequence of SEQ ID NO: 1 that binds to an integrin such as αᵥβ₃ or α₅β₁. The invention also provides for a polynucleotide sequence encoding a peptide comprising the amino acid sequence of SEQ ID NO: 2 that consists of residues 257-272 of SEQ ID NO: 1 or a peptide comprising the amino acid sequence of SEQ ID NO: 19 that consists of residues 285-291 of SEQ ID NO: 1.

CTGF is known to induce extra cellular matrix production and to stimulate fibroblast and smooth muscle cell proliferation and chemotaxis. Thus, CTGF peptides or CTGF agonists may be effective in stimulating wound healing. The invention provides for methods of inducing wound healing comprising administering CTGF peptides, such as CTGF[257-272] or CTGF[285-291], or CTGF agonists, in an effective amount to a mammal in need. CTGF agonists include molecules that stimulate or enhance cellular signaling induced by CTGF binding to an integrin such as αᵥβ₃ or α₅β₁. For example, the invention provides for-peptides comprising a fragment of SEQ ID NO: 1 that stimulate CTGF and integrin binding or stimulate CTGF biological activity. CTGF agonists also include molecules that increase CTGF activity, including but not limited to CTGF-induced extra cellular matrix production, cell proliferation, cell migration, cell cycle progression, cell differentiation, cell adhesion, chemotaxis, apoptosis, gene transcription and ion transport. The invention further provides for methods of stimulating the binding of CTGF to an integrin, such as αᵥβ₃ or α₅β₁., comprising administering a peptide of the invention in an effective amount. The invention also provides for methods of stimulating CTGF biological activity comprising administering a peptide of the invention in an effective amount.

The invention also provides for compositions comprising a CTGF agonist that stimulates or enhances cellular signaling induced by CTGF binding to an integrin such as αᵥβ₃ or α₅β₁ and a carrier. For example, administration of the CTGF peptide or CTGF agonists may be useful for inducing or accelerating wound healing and repairing connective tissue, bone or cartilage. In addition, administration of the CTGF peptides or CTGF agonists may be effective in inducing the formation of bone, tissue or cartilage in disorders such as osteoporosis, osteoarthritis, hypertrophic scars, burns, vascular hypertrophy or wound healing.

The invention provides kits that are useful for stimulating or enhancing cellular signaling induced by CTGF binding to an integrin such as αᵥβ₃ or α₅β₁. For example, the invention provides for kits useful for stimulating wound healing or formation of bone, tissue or cartilage in a mammal in need, wherein the kit comprises a peptide of the invention and a set of instructions for administering the peptide to, e.g., a mammal, such as a human in need.

The invention provides for molecules which inhibit the binding of CTGF and an integrin, such as αᵥβ₃ or α₅β₁, or act as an inhibitor of cellular signaling induced by CTGF binding with an integrin, such as αᵥβ₃ or α₅β₁. Such inhibitory molecules include antibodies and small molecules that specifically bind to a peptide comprising a fragment of the amino acid sequence of SEQ ID NO: 1, a peptide comprising the amino acid sequence of SEQ ID NO: 2 or a peptide comprising the amino acid sequence of SEQ ID NO: 19. The invention also provides for antibodies and small molecules that specifically bind to peptides that block CTGF binding sites for integrins and thereby inhibit CTGF biological activity. The invention also provides peptides that act as inhibitors of cellular signaling induced by CTGF binding to αᵥβ₃ or α₅β₁. These inhibitory peptides include those that inhibit the binding of CTGF and an integrin; such as αᵥβ₃ or α₅β₁. These inhibitory peptides also include those which inhibit CTGF biological activity including but not limited to CTGF-induced extra cellular matrix production, cell proliferation, cell migration, cell cycle progression, cell differentiation, cell adhesion, chemotaxis, apoptosis, gene transcription and ion transport.

The invention further provides for methods of inhibiting the binding of CTGF to an integrin, such as 60 ᵥβ₃ or α₅β₁, comprising administering an effective amount of an inhibitory peptide, antibody or small molecule of the invention. The invention also provides for methods of inhibiting CTGF biological activity; comprising administering an inhibitory peptide, an antibody or small molecule of the invention in an effective amount.

The invention provides for methods of inhibiting CTGF binding to an integrin, such as αᵥβ₃ or α₅β₁, comprising administering an effective amount of an antibody, small molecule or peptide that specifically binds SEQ ID NO: 2 or SEQ ID NO: 19 in an effective amount, wherein the binding to the sequence of SEQ ID NO: 2 or SEQ ID NO: 19 inhibits the interaction between CTGF and an integrin. The invention also provides methods of identifying agents that modulate CTGF activities comprising the steps of contacting CTGF and an integrin, such as αᵥβ₃ or α₅β₁, in the presence and absence of a test agent, determining the CTGF activity in the presence and absence of the test agent, and comparing the CTGF activity in the presence of the test agent to the activity in the absence of the test agent to identify agents that modulate CTGF activity, wherein a modulator that is a CTGF inhibitor reduces CTGF activity and a modulator that is a CTGF agonist increases CTGF activity.

The invention provides for methods of treating CTGF-related disorders such as fibroproliferative disorders, connective tissue disorders, hyperproliferative disorders and angiogenesis-related disorders as described herein. The methods comprise administering a molecule that inhibits CTGF binding to an integrin such as αᵥβ₃ or α₅β₁ in an effective amount, wherein the CTGF binding to an integrin promotes the CTGF-related disorder. The invention also provides for compositions comprising a molecule that inhibits CTGF binding to an integrin such as αᵥβ₃ or α₅β₁ and a carrier for treatment of a CTGF-related disorder. The invention also provides kits useful in treating CTGF-related disorders, wherein the kit comprises a peptide of the invention and a set of instructions for administering the peptide to, e.g., a mammal, such as a human in need.

Fibrosis occurs in multiple organs and is a major disease area that lacks effective therapies for prevention or treatment. Chronic fibrosis most commonly affects the liver, pancreas, lung, kidney, heart, and skin while acute fibrosis is associated with the formation of scar tissue in response to surgery or trauma (stroke, heart attack, burns, radiation chemotherapy). More than 90% of surgical patients are affected by collagen-rich adhesions that entrap adjacent tissues and prevent proper function or healing.

CTGF is present and frequently over-expressed and co-expressed with TGF-β in fibrotic skin disorders such as systemic sclerosis, localized skin sclerosis, keloids, scar tissue, eosinic fasciitis, nodular fasciitis, and Dupuytren's contracture. CTGF mRNA and/or protein are over-expressed in fibrotic lesions of major organs and tissues including the liver, kidney, lung, cardiovascular system, pancreas, bowel, eye, and gingiva. CTGF is also over-expressed in the stromal compartment of melanoma as well as mammary, pancreatic and fibrohistiocytic tumors that are characterized as having significant connective tissue involvement. Collectively, these data support a role for CTGF as a downstream mediator of some of the fibrogenic actions of TGF-β1. CTGF may be a molecular target-for therapeutic intervention in fibrotic diseases. In addition, CTGF plays an important role in the deposition of matrix components and activation of growth factors that support accompanying fibrogenesis.

In fibrotic liver, CTGF mRNA and protein are produced by fibroblasts, myofibroblasts, hepatic stellate cells (HSCs), endothelial cells, and bile duct epithelial cells. CTGF is also produced at high levels in hepatocytes during cytochrome P-4502E1-mediated ethanol oxidation. CTGF expression in cultured HSCs is enhanced following their activation or stimulation by TGF-β while exogenous CTGF is able to promote HSC adhesion, proliferation, locomotion, and collagen production. Collectively, these data suggest that during initiating or downstream fibrogenic events in the liver, production of CTGF is regulated primarily by TGF-β in one or more cell types and that CTGF plays important roles in HSC activation and progression of fibrosis .(Rachfal & Brigstock, Hepatol Res. 26(1):1-9, 2003)

CTGF expression and action has been linked to pancreatic fibrosis, which is a frequent feature of chronic pancreatitis. Fibrosis is a major feature of chronic alcoholic pancreatitis which, like acute pancreatitis, is associated with long-term heavy alcohol consumption (Slauja et al., Pancreas; 27: 327-31, 2003). CTGF and, TGF-β mRNA are enhanced in human acute necrotizing pancreatitis tissue samples compared with normal controls (di Mola et al., Ann Surg 230:63-71, 1999). In a model of acute necrotizing pancreatitis in rats, mRNA for CTGF, TGF-β, and collagen type 1 were concomitantly enhanced (di Mola et al., Ann Surg 230:63-71, 1999). In patients undergoing surgery for chronic pancreatitis; there was coordinate over-expression of CTGF, TGF-β, TGF-β receptors and collagen type I (di Mola et al., Ann Surg 235:60-7, 2002).

Thus, inhibition of CTGF binding to an integrin such as αᵥβ₃ or α₅β₁, may be useful for treating or preventing fibroproliferative disorders and connective tissue disorders. Fibroproliferative disorders include but are not limited to chronic and acute fibrosis, diabetic nephropathy, glomerulonephritis, proliferative vitreoretinopathy, liver cirrhosis, biliary fibrosis, myelofibrosis, postradiation fibrosis and retinopathy. Connective tissue disorders, such as rheumatoid arthritis, scleroderma, myelofibrosis, and hepatic, and pulmonary fibrosis also may be treated by inhibiting CTGF binding to an integrin, such as α_{vβ}₃ or α₅β₁.

Angiogenesis is required for neovascularization during embryogenesis, placentation, tumor growth and metastasis. Inhibition of angiogenesis may be a valuable new approach to cancer therapy because avascular tumors are severely restricted in their growth potential due to a blood supply. Endothelial cells are an important target of CTGF actions and therefore CTGF may play a role in regulating endothelial cell function and angiogenesis. CTGF promotes endothelial cell growth, migration and adhesion in vitro and is transcriptionally activated in endothelial cells in response to basic fibroblast growth factor (bFGF) or vascular endothelial growth factor (VEGF) (Wunderlich et al., Graefes Arch Clin Exp Ophthalmol; 238: 910-5, 2000; Shimo et al., J Biochem (Tokyo); 126: 137-45, 1999; Suzuma et al., J Biol Chem; 275: 40725-31, 2000). Endothelial cell proliferation and migration in vitro is reduced by antagonists of CTGF production or action (Shimo et al., J Biochem (Tokyo) 124: 130-40, 1998). Inhibition of endogenous expression of connective tissue growth factor by its antisense oligonucleotide and antisense RNA suppresses proliferation and migration of vascular endothelial cells. The expression pattern of CTGF in endothelial cells of vessels in situ supports a role for CTGF in normal endothelial homeostasis, as well as participating in angiogenesis during embryonic development, placentation, tumor formation, fibrosis, and wound healing. CTGF is intrinsically active in in vivo assays for angiogenic activity (Shimo et al., supra., Babic et al., supra.). However, CTGF also regulates the production and/or activity of other angiogenic molecules (e.g. BFGF, VEGF) that affect the integrity or stability of the ECM (e.g. collagen, matrix metalloproteases (MMPs), tissue inhibitors of MMPs) (Inoki et al., Faseb J; 16: 219-21,2002; Hashimoto et al., J Biol Chem; 277: 36288-95, 2002; Kondo et al., Carcinogenesis; 23: 769-76, 2002). Furthermore, endothelial cells are known to express integrin αᵥβ₃ and integrins are known to play an important role in the process of angiogenesis. Endothelial cells express integrin α₅β₁ , which has been shown to bind to NOV and to regulate angiogenesis (Ellis et al., Vasc Res. 40(3):234-43, 2003; Lin et al., J Biol Chem. 27: 278(26):24200-8, 2003). Therefore, through its paracrine action as a product of cells such as fibroblasts or smooth muscle cells or through its autocrine action as an endothelial cell product CTGF participates in a variety of direct and indirect angiogenic pathways.

Thus inhibition of the binding of CTGF to an integrin such as αᵥβ₃ or integrin α₅β₁ may be useful for treating or preventing angiogenesis and tumor growth. Therapeutic compositions of the invention may be effective in adult and pediatric oncology including in solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma; brain cancers including intrinsic brain tumors, neuroblastoma, astrocytic brain tumors, gliomas, metastatic tumor cell invasion in the central nervous system, bone cancers including osteomas, sarcomas including fibrosarcoma and osteosarcoma, skin cancers including malignant melanoma, tumor progression of human skin keratinocytes, squamous cell carcinoma, basal cell carcinoma, hemangiopericytoma and Karposi's sarcoma.

CTGF mRNA was overexpressed in 80% of pancreatic cancer tissues tested and its level was correlated to the degree of fibrosis in those cancers. CTGF mRNA was also overexpressed in nude mouse xenograft tumors CTGF was produced predominantly by fibroblasts and was implicated in the development of a desmoplastic stromna (Wegner et al., Oncogene 18:1073-80, 1999). In human hepatocarcinoma, CTGF is overexpressed as compared to the surrounding normal tissue (Hirasaki et al., Hepatol Res. 26: 19(3):294-305, 2001).

Inhibitors of binding between CTGF and an integrin, such as αᵥβ₃, binding may be effective for treating or preventing disorders associated with sustained scarring of blood vessels. Such disorders include artheroscelosis, hypertension, systemic sclerosis, inflammatory bowel disease, and Chrohn's disease.

CTGF is also known to induce cell proliferation. Thus, inhibition of the binding between CTGF and an integrin, such as αᵥβ₃ or α₅β₁, may be useful for treating or preventing hyperproliferative disorders. Hyperproliferative disorders include but are not limited to precancerous and hyperplastic conditions, cancers as listed herein, psoriasis, contact dermatitis, immune disorders and inflammatory disorders such as arthritis, inflammatory bowel disease and Chrohn's disease. Hyperproliferative disorders also include infertility and disorders within the female reproductive tract.

### CCN Family Members and Integrin Binding

It is known that forms of CTGF, (CTGF₁₋₄, CTGF₃₋₄, and CTGF₄) can support adhesion of multiple cell types including fibroblasts, endothelial cells, myofibroblasts, and epithelial cells (Ball et al., J Endocrinol; 176: R1-R7, 2003; Ball et al., Reproduction 125:271-284, 2003). This is consistent with the finding that CTGF is a matrix-associated protein and is presented to target cells as an adhesive substrate (Kireeva et al., Exp Cell Res; 233: 63-77, 1997). As described herein, CTGF utilizes integrins such as αᵥβ₃ or α₅β₁ as adhesive receptors (See Example 3-6 and 9 and 11). Full-length CTGF and CYR61 (CCN family members) are ligands of, and bind directly to, specific integrins in a cell-specific manner (Lau et al., Exp Cell Res; 248: 44-57, 1999). Fibroblast adhesion to CYR61 or CTGF results in a cascade of adhesive signaling events that include formation of filopodia and integrin focal complexes, and activation of focal adhesion kinase, paxillin, Rac, and mitogen-activated protein kinase (Chen et al., J Biol Chem; 276: 10443-52, 2001). This view is substantiated by the ability of integrins to transduce extracellular binding events into intracellular signaling cascades (Chen et al., J Biol Chem; 276: 10443-52, 2001; Chen et al., J Biol Chem; 276: 47329-37, 2001).

Although CCN proteins do not contain an RGD sequence, RGD peptides: can be effective in inhibiting integrin binding by CCN proteins such as CTGF, CYR61 or NOV. For example, the binding of integrin αᵥβ₃ to either CTGF or CYR61, or the binding of integrin α₅β₁ to either NOV or CTGF, is RGD-sensitive (Lau et al., Exp Cell Res; 248: 44-57, 1999; Babic et al., Mol Cell Biol; 19: 2958-66, 1999; Chen et al., J Biol Chem; 276: 47329-37, 2001; Leu et al., J Biol Chem; 277: 46248-55, 2002; Chen et al., J Biol Chem; 276: 10443-52, 2000; Lin et al., 278(26): 24200-8, 2003). These data indicate that there is an RGD-induced conformational change in each integrin subtype that precludes subsequent binding by CTGF. CYR61 or NOV. Although CYR61 and CTGF can engage a variety of integrin subtypes, both molecules can engage integrin αᵥβ₃ on fibroblasts, HSCs, HUVECs, and breast cancer cells (Babic et al., Mol Cell Biol; 19: 2958-66, 1999; Chen et al., J Biol Chem; 276: 47329-37, 2001; Leu et al., J Biol Chem; 277: 46248-55, 2002; Chen et al., J Biol Chem; 276: 10443-52, 2000; Menendez et al., Endocrine-Related Cancer; 10: 139-50; 2003). In fact, the aggressiveness of breast cancer is directly linked to the molecular association of CYR61 with integrin αᵥβ₃ on breast cancer cells and this has major implications regarding novel therapeutic approaches (Menendez et al., Endocrine-Related Cancer; 10: 139-50, 2003). To this end, it is critical to uncover the molecular basis of the interaction between CCN proteins and their respective integrin receptors. EDTA-sensitive (i.e. integrin-mediated) cell adhesion resides in module 4 (Ball et al., J Endocrinol; 176: R1-R7, 2003).

This observation is considerably refined by showing that the CTGF peptide IRTPKISKPIKFELSG (SEQ ID NO: 2), corresponding to residues 257-272 in CTGF₄, inhibits adhesion of hepatic stellate cells (HSCs) to CTGF₄ and is able to support HSC adhesion via integrin αᵥβ₃. (See Example 3) The peptide also binds strongly and directly to integrin αᵥβ₃ in a cell-free binding assay. (see Example 4). In addition, the data in Examples 9-11 demonstrate that CTGF also binds to integrin α₅β₁. The CTGF peptide GVCTDGR (SEQ ID NO: 19), corresponding to residues 285-291 in CTGF₄ (SEQ ID NO: 1), inhibited adhesion of pancreatic stellate cells (PSCs) to CTGF₄ and supported PSC adhesion via integrin α₅β₁. (See Example 11) The peptide also bound strongly and directly to integrin α₅β₁ in a cell-free binding assay (See Example 11). Therefore, there are at least two integrin binding sites exist within this highly defined region of CTGF₄.

### Assays for CTGF Activity

Assays for measuring CTGF-induced cellular activities are known in the art. Examples of such assays are described herein.

It is known that CTGF supports the adhesion of multiple cell types including fibroblasts, endothelial cells, myofibroblasts, and epithelial cells (Ball et al., J. Endocrinology 176: R1-R7, 2003). Assays that measure CTGF-mediated cell adhesion may be used to determine if CTGF peptides or CTGF modulators are functional. For example, cells are suspended in serum free medium containing 0.5% BSA and plated on CTGF precoated plates. The cells are incubated for 20 minutes at 37° C., and then washed three times with PBS. Adherent cells are fixed with 10% formalin and stained by addition of 100 μl CyQUANT GR dye/cell lysis buffer to each sample well and incubating for 5 minutes at room temperature, protected from light. The number of adherent cells are quantitated by measuring the fluorescence intensity using a micro-plate reader at Ex/Em: 480/520 nm.

CTGF is known to stimulate DNA synthesis in pig endometrial cells, 3T3 cells (Brigstock et al., J. Biol. Chem., 275: 24953-61) and HSCs and PSCs (see Example 11). DNA synthesis may be measured using [³H] thymidine incorporation or BrdU labeling. For example, quiescent cell cultures using of HSCs or human umbilical endothelial cells (HUVECs) are stimulated with 0-100 ng/ml of each CTGF peptides or CTGF modulators in the presence of [³H]thymidine. TCA-insoluble cpm is evaluated 24-48 hours later. DNA synthesis also is evaluated by measuring cell proliferation using MTT assays or cell counting. Use of assays that measure DNA synthesis are methods of determining if the CTGF peptide or CTGF modulators that affect CTGF activity.

CTGF is also known to induce transdifferentiaion of corneal epithelial cells into α small muscle actin (αSMA)-expressing myofibroblasts. For example, cells are cultured for 24 hours in the presence and absence of CTGF peptide or CTGF modulator. Subsequently, the treated cells are stained for αSMA. CTGF activity also may be analyzed by measuring for a classic fibrogenic response as described in Leask et al., Mol. Pathol; 54: 180-183, 2001. For example, CTGF peptide or CTGF modulator are administered into the subcutaneous region of the neck of 3 day old mice for 7 consecutive days. Fourteen days after the last injection, the mice are sacrificed and the injected areas are stained for αSMA.

The CTGF[257-272] peptide is located within the heparin binding domain of the full length CTGF polypeptide. Heparin-affinity chromatography using high resolution HPLC columns may be used to assess the heparin binding properties of a CTGF peptide or CTGF modulator. Non-heparin binding molecules and weakly non-heparin-binding molecules pass directly through the column. Weakly heparin-binding molecules are eluted with approx 0.2-0.6 NaCl whereas molecules in which the heparin-binding properties resemble those of the CTGF parental protein require 0.7-0.8 M NaCl for elution. Proteins are detected at 214 nm using an in-line UV monitor. The heparin-binding properties may also be evaluated based on their ability to bind to [³H]heparin as described in Brigstock et al., J. Biol. Chem., 272: 20275-82, 1997. Briefly, CTGF peptide or CTGF modulator are absorbed to nitrocellulose and incubated for 3 hours at room temperature in the presence of 10 μCi/ml [³H]heparin. After washing, the individual dots are counted for ³H. To ensure that the binding of the proteins to nitrocelluose or plastic is not compromised by the protein itself, it is verified that the protein has quantitatively absorbed to the substrate by ELISA using CTGF antibody.

CTGF is also known to stimulate dose-dependent production of TIMPs-1, -2, -3 and -4 in fibroblasts (Wang et al., Wound Repair Regen., 11 :220-9, 2003). To measure TIMP production, HSCs and HUVECs are treated with CTGF peptide or CTGF modulator for up to 24 hours prior to RNA isolation and RT-PCR for TIMPs1-4 as described in Wang et al., supra. Effects on angiogenesis may also be assessed to analyze CTGF activity using methods known in the art. For example, the aortic ring. assays are carried in which isolated rat aorta is cut into segments and placed in culture with Matrigel. CTGF peptide or CTGF modulator are added and the explants monitored for outgrowth of endothelial cells over the next 14 days. The number and length of vessel-like outgrowth that stain with endothelium-specific markers such as fluorescein-labeled BSL-1 are measured as described in Go et al., Methods Mol. Med. 85: 59-64, 2003.

### Peptides

The CTGF peptides, including CTGF[257-272] and CTGF[285-291], can be prepared in a number of conventional ways. The short peptides sequences may be prepared by chemical synthesis using standard means. Particularly convenient are solid phase techniques. (see, e.g., Erikson et al., The Proteins (1976) v. 2, Academic Press, New York, p. 255). Automated solid phase synthesizers are commercially available. In addition, modifications in the sequence are easily made by substitutions addition or omission of appropriate residues. For example, a cysteine residue may be added at the carboxy terminus to provide a sulfhydryl group for convenient linkage to a carrier protein, or spacer elements, such as an additional glycine residue, may be incorporated into the sequence between the linking amino acid at the C-terminus and the remainder of the peptide. The CTGF peptides of the present invention can also be produced by recombinant techniques. The coding sequence for peptides of this length. can easily be synthesized by chemical techniques, e.g., the phosphotriester method described in Matteucci et al., J Am. Chem. Soc., 103: 3185, 1981.

The invention also provides for mutant or variant CTGF peptides with one or more conservative amino acid substitutions that do not affect the biological and/or immunogenic activity of the polypeptide. Alternatively, the CTGF peptides of the invention are contemplated to have conservative or nonconservative amino acids substitutions which may alter (increase or decrease) the ability to bind to integrin αᵥβ3 or induce CTGF activities. The term “conservative amino acid substitution” refers to a substitution of a native amino acid residue with a normative residue, including naturally occurring and nonnaturally occurring amino acids, such that there is little or no effect on the polarity or charge of the amino acid residue at that position. For example, a conservative substitution results from the replacement of a non-polar residue in a polypeptide with any other non-polar residue. Further, any native residue in the peptide may also be substituted with alanine, according to the methods of “alanine scanning mutagenesis” (See Example 7); Naturally occurring amino acids are characterized based on their side chains as follows: basic: arginine, lysine, histidine; acidic: glutamic acid, aspartic acid; uncharged polar: glutamine, asparagine, serine, threonine, tyrosine; and non-polar: phenylalanine, tryptophan, cysteine, glycine, alanine, valine, proline, methionine, leucine, norleucine, isoleucine. General. rules for amino acid substitutions are set forth in Table 1 below.

TABLE 1
Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asn |
| Gly | Pro, Ala | Ala |
| His | Asn, Gln, Lys, Arg | Arg |
| Ile | Leu, Val, Met, Ala, Phe, | Leu |
| Leu | Norleucine, Ile, Val, Met, | Leu |
| Lys | Arg, 1,4 Diaminobutyric | Arg |
| Met | Leu, Phe, Ile | Leu |
| Phe | Leu, Val, Ile, Ala, Tyr | Arg |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |

### Inhibition of CTGF Interaction with an Integrin

The present invention provides for methods of identifying modulators (antagonists/inhibitors or agonists/stimulators) of CTGF and integrin binding such as αᵥβ₃ and α₅β₁, or CTGF activities comprising contacting an antibody or small molecule with CTGF or a CTGF peptide and measuring αᵥβ₃ binding and/or activity elicited by the interaction in the presence and absence of these small molecules or antibodies. The small molecules can be naturally occurring medicinal compounds or derived from combinational chemical libraries. In certain embodiments, CTGF modulators may be a protein, peptide, carbohydrate, lipid, or small molecule that interacts with the CTGF to regulate its activity or inhibit integrin binding.

### Antibodies

The present invention provides for antibodies and antibody fragments that bind to CTGF, in particular CTGF[257-272] (SEQ ID NO: 2) or CTGF[285-291] (SEQ ID NO: 19). The antibodies of the invention include those that inhibit binding of CTGF to an integrin'such as αᵥβ₃ or α₅β₁. The antibodies also include those that inhibits CTGF biological activity. The invention also provides for antibodies that bind to the CTGF polypeptide and induce a conformational change that prevents CTGF from binding to an integrin such as α_{vβ}₃ or α₅β₁.

The antibodies may be polyclonal including monospecific polyclonal, monoclonal (mAbs), recombinant, chimeric, humanized such as CDR-grafted, human, single chain, and/or bispecific, as well as fragments, variants or derivatives thereof. Antibody fragments include those portions of the antibody which bind to an epitope on the CTGF peptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions. Methods for making antibodies specific for CTGF peptides are described in Brigstock et al., J. Biol. Chem., 275: 24953-61, 1997.

Polyclonal antibodies directed toward CTGF generally are produced in animals (e.g., rabbits or mice) by means of multiple subcutaneous or intraperitoneal injections of the CTGF and an adjuvant. It may be useful to conjugate a CTGF peptide to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for CTGF antibody titer.

Monoclonal antibodies directed toward CTGF are produced using any method which provides for the production of antibody molecules by continuous cell lines in culture. Examples of suitable methods for preparing monoclonal antibodies include the hybridoma methods of Kohler et al., Nature, 256:495-497 (1975) and the human B-cell hybridoma method, Kozbor, J. Immunol., 133:3001 (1984); Brodeur et al., Monoclonal Antibody Production Techniques and Applications, pp. 51-63 (Marcel Dekker, Inc., New York, 1987). Also provided by the invention are hybridoma cell lines which produce monoclonal antibodies reactive with CTGF peptide polypeptides.

Antibodies which specifically bind to CTGF may be used to provide reagents for use in diagnostic assays for the detection of the CTGF polypeptide in various body fluids. In another embodiment, the CTGF peptide may be used as antigens in immunoassays for the detection of CTGF in various patient tissues and body fluids including, but not limited to: ambiotic fluid, blood, serum, ear fluid, spinal, fluid, sputum, urine, lymphatic fluid and cerebrospinal fluid. The antigens of the present invention may be used in any immunoassay system known in the art including, but not limited to: radioinmmunoassays, ELISA assays, sandwich assays, precipitin reactions, gel diffusion precipitin reactions, immunodiffusion assays, agglutination assays, fluorescent immunoassays, protein A immunoassays and immunoelectrophoresis assays.

For diagnostic applications, antibodies that specifically bind CTGF may be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase, or horseradish peroxidase (Bayer et al., Meth. Enz., 184:138-163, 1990).

### Compositions

Pharmaceutical compositions comprising a CTGF peptide or CTGF modulator of the invention are provided. The pharmaceutical compositions may comprise one or more additional ingredients such as pharmaceutically effective carriers. Dosage and frequency of the administration of the pharmaceutical compositions are determined by standard techniques and depend, for example, on the weight and age of the individual, the route of administration, and the severity of symptoms. Administration of the pharmaceutical compositions may be by routes standard in the art, for example, parenteral, intravenous, oral, buccal, nasal, pulmonary, rectal, or vaginal.

The present invention also provides kits to facilitate single-dose or multiple dose administrations of a peptide of the invention, along with a set of instructions for administration thereof. The instructions are provided in any form suitable under the circumstances. In some embodiments, the kits may each contain both a first container having a dried protein and a second container having an aqueous formulation. Such formulation include physiological saline, solution, neutral buffered saline, artificial cerebrospinal fluid. The optimal pharmaceutical formulation will be determined by one skilled in the art depending upon, for example, the intended route of administration, delivery format, and desired dosage. See for example, Remington's Pharmaceutical Sciences, 18th Ed., A. R. Gennaro, ed., Mack Publishing Company (1990). Also included within the scope of this invention are kits containing single- and multi-chambered syringes: (e.g., liquid syringes and lyosyringes) pre-filled with at least one peptide according to the invention.

### BRIEF DESCRIPTION OF FIGURES

FIG. 1 demonstrates integrin-mediated cellular adhesion to CTGF. HSCs were plated in wells coated with CTGF isoforms (CTGF₁₋₄, CTGF₃₋₄ and CTGF₄) (2 μg/ml) or laminin (LN, 1 μg/ml),or vitronectin (VN, 4 μg/ml) at 4° C. for 16 h. After incubation at 37° C. for 20 min, adherent cells were washed, fixed and stained with CyQUANT GR dye, followed by measuring the fluorescence at EX/EM: 480/520. Panel A: Cell suspensions were incubated in the presence or absence (N/A) of 10 mM EDTA for 30 minutes prior to plating. Adhesion of HSC to the CTGF isoforms was affected by EDTA. Panel B: 1 mM GRGDsp, GRGEsp, 2 μM Echistatin or their vehicle buffer alone (N/A) was individually present in the medium for 30 min prior to plating, both GRGDsp peptide and Echistatin were able to block CTGF isoform-mediated and, VN-mediated cell adhesion. Panel C: Echistatin exhibited a dose-dependent inhibition of CTGF₄-mediated and VN-mediated cell adhesion. Data shown for all panels are mean±S.D. of quadruplicate determinations and are representative of three experiments.

FIG. 2 demonstrates that CTGF₄ was involved in integrin αᵥβ₃-mediated HSC adhesion. Cell adhesion assays were performed on the 96-well costar plate coated with CTGF₄ (2 μg/ml) following by (Panel A) incubation with 10 mM EDTA for 30 minutes or addition of 10 mM Ca²⁺, Mn²⁺ or Mg²⁺ or their vehicle buffer alone (N/A) to the cell suspension prior to plating. Panel B: the cells were treated with 25 μg/ml LM609 (anti-αᵥβ₃) or mouse IgG (25 μg/ml) or vehicle buffer alone (N/A) at room temperature for 30 minutes. Data shown for all panels are mean±S.D. of quadruplicate determinations and are representative of three experiments

FIG. 3 demonstrates integrin αᵥβ₃ binding specific for CTGF[257-272]. Panel A: microtiter wells were coated-with different concentrations of each CTGF isoform or VN at 4° C. for 16 hours, and blocked with 2% BSA for 2 h. 1 μg/ml integrin α_{vβ}₃ was added to the wells and allowed to bind for 3 h at room temperature. Bound integrin αᵥβ₃ was detected with anti-αᵥβ₃, followed by HRP-conjugated secondary antibody and 3,3′, 5,5′-tetramethyl benzidine as the substrate. Panel B: microtiter wells were coated with 2 μg/ml CCN2 or 4 μg/ml VN. 1 μg/ml integrin αᵥβ₃ was added to the wells alone or following by incubation with 35 μM CTGF[257-272] for 1 hour. The inhibitory effect of peptide on binding of integrin αᵥβ₃ with CTGF₄ was quantified by ELISA. Data shown for all panels are mean±S.D. of quadruplicate determinations and are representative of three experiments.

FIG. 4 demonstrates that CTGF adhesion to PSC is mediated through integrin α₅β₁. Panel A: Microtiter wells were precoated with 2 μg/ml of CTGF₁₋₄, CTGF₃₋₄ and CTGF₄ (denoted as CCN2₁₋₄, CCN2₃₋₄, CCN2₄ in figure) or 2 μg/ml fibronectin (FN) at 4° C. for 16 hours and then blocked with PBS and 1% BSA for 1 hour. PSC (2.5×10⁵ cells/ml) were preincubated in serum-free medium for 30 minutes in vehicle buffer (no add) or EDTA (5 mM) prior to the addition to individual wells at 50 μl/well. The adherent cells were stained and quantified by measuring the fluorescence intensity at an excitation of 485 nm and an emission of 530 nm. Panel B: PSC adhesion was measured as described for Panel A. PSCs were preincubated with EDTA (5 mM), either alone or in combination with Ca²⁺ (10 mM) or Mg²⁺ (10 mM), for 30 minutes. Panel C: PSC adhesion was measured as described for Panel A. PSC were preincubated with 25 μg/ml of anti-α₅ or anti-β₁ monoclonal antibody for 30 minutes and subsequently the cells were added to wells precoated with CTGF₄(2 μg/ml) (denoted as CCN2₄ in the Figure), fibronectin (FN; 2 μg/ml) or vitronectin (VN; 4 μg/ml). Panel D: PSC adhesion was measured as described for Panel C. PSC were preincubated at 37° C. for 30 minutes with vehicle buffer (no add), anti-α₅β₁ (Temicula, Calif.) antibody or mouse IgG (25 μg/ml). The data in all panels is the mean±S.D. of quadruplicate determinations of results for each experiment with the data reflecting the results and are representative of three experiments.

FIG. 5 demonstrates that integrin α₅β₁ binds to CTGF₄ directly in cell-free systems. Panel A: 2 μg/ml integrin α₅β₁ was added to 4 μg/ml of CTGF₁₋₄, CTGF₃₋₄, CTGF₄ or CTGF₃ (denoted as: CCN2₁₋₄, CCN2₃₋₄, CCN2₄ or CCN2₃ in the figure) diluted in 1 ml NP40 buffer, prior to immunoprecipitation with a polyclonal rabbit anti-CCN2 antibody or normal IgG. The samples were analyzed by immunoblotting with anti-human α₅β₁ (Temicula, Calif.). :Panel B Microtiter wells precoated with 2 μg/ml of CTGF₁₋₄, CTGF₃₋₄, CTGF₄ or CTGF₃ (dentoed as CCN2₁₋₄, CCN2₃₋₄, CCN2₄ or CCN2₃ in the figure) or FN were incubated with 1 μg/ml integrin α₅β₁ in the blocking solution described above. The solid phase ELISA assay was carried out as described in Example 3,and CTGF binding to α₅β₁ was measured at absorbance 450. Panel C: Microtiter wells precoated with 2 μg/ml CTGF₄ (denoted as CCN2₄ in the figure) or FN (2 μg/ml) were incubated with a in the presence and absence of 5 mM EDTA alone in combination with 10 mM Ca⁺⁺ or 10 mM Mg⁺⁺. The solid phase ELISA assay was carried out as described in Example 3 and CTGF binding to α₅β₁ was measured at absorbance 450. The data in all panels is mean±S.D. of quadruplicate determinations for each experiment with the data representing the results of three experiments.

FIG. 6 demonstrates that peptide CTGF [285-291]) contains a α₅β₁ binding site. Panel A: PSC adhesion assays were performed as described in Example 3. 96-well plates were separately coated with 2 μg/ml of one of synthetic peptides P1-P11 (SEQ ID NOS: 26, 2, 27, 28, 9, 29, 30, 31, 32, 33 and 34, respectively) that span the 103 c-terminal residues of CTGF. The adherent cells were stained and quantified by measuring the fluorescence intensity at an excitation of 485 nm and an emission of 530 nm. Panel B: Peptide P2 (10 μm), P5 (10 μm) or vehicle buffer alone (no add) were added individually to PSC suspensions for 30 minutes at room temperature. The cells were plated on microtiter wells that had been precoated with 2 μg/ml CTGF₄ (denoted as CCN2₄ in the figure), or 4 μg/ml fibronectin (FN). The adherent cells were stained and quantified by measuring the fluorescence intensity at an excitation of 485 nm and an emission of 530 nm. Panel C: Microtiter wells were coated with 2 μg/ml CTGF₄ (denoted as CCN2₄ in the figure), or 4 μg/ml fibronectin (FN), then incubated with 1 μg/ml integrin α₅β₁ alone (no add) or in the presence of 35 μM peptide P2 or 35 μM peptide P5 for 1 hour. The CTGF₄ binding of integrin α₅β₁ was quantified by the solid-phase ELISA as described in Example 3, and integrin α₅β₁ binding was measured at absorbance 450. Panel D, Microtiter wells coated individually with CTGF₄ (denoted as CCN2₄ on the figure), peptide M₁ (SEQ ID NO: 10; 8 μg/ml), peptide M2 (SEQ ID NO: 11; 8 μg/ml), peptide M3 (SEQ ID NO: 12; 8 μg/ml) or peptide M4 (SEQ ID NO: 13; 8 μg/ml) at 4° C. for 16 hours. Then, peptide binding to integrin α₅β₁ was analyzed using the solid-phase ELISA assay described in Example 3. Integrin α₅β₁ was measured at absorbance 450. The data for all panels is the mean±S.D. of quadruplicate determinations, for each experiment with the data representing the results of three experiments.

### DETAILED DESCRIPTION

The following examples illustrate the invention wherein Example 1 describes production of bioactive proteolytic fragments of CTGF, Example 2 describes integrin-mediated adhesion of activated HSC to CTGF, Example 3 describes CTGF₄, Example 4 describes binding of integrin αᵥβ₃ to CTGF[257-272] in CTGF₄, Example 5 describes adhesion of activated HSC to CTGF requires cell surface heparin sulphate proteoglycans (HSPG), Example 6 describes site-directed mutagenesis of residues 257-272 of CTGF, Example 7 describes development of short peptide antagonists between CTGF and integrin αᵥβ₃, and Example 8 describes development of peptide antibodies that block CTGF-αᵥβ₃ binding. Example 9 demonstrates adhesion of activated PSC to CTGF is mediated via integrin α₅β₁ and HSPG. Example 10 describes stimulation of PSC migration, proliferation and collagen synthesis are induced by CTGF. Example 11 describes binding of α₅β₁ to peptide CTGF[285-291]. The CTGF properties may be demonstrated in any cell type which express CTGF receptors or is response to CTGF. The Applicants do not intend to be limited by the following examples.

### EXAMPLE 1

### Production of Stable Bioactive Proteolytic Fragments of CTGF

Two N-terminally truncated forms of CTGF are naturally found in pig uterine secretory fluid. (Brigstock et al., J Biol Chem; 272: 20275-82 1997). The N-terminal amino sequences of each protein are identical to, respectively, residues 247-262 and 248-259 of the predicted sequence of human CTGF. These low mass CTGFs are also present in uterine secretions of the mouse (Surveyor et al., Biol Reprod; 59: 1207-13, 1998). Conditioned medium from cultured fibroblasts contained 10-12 kDa heparin-binding immunoreactive forms of CTGF that are mitogenic (Steffen et al., Growth Factors; 15: 199-213, 1998). These proteins are soluble bioactive 10-20 kDa CTGF proteins generated through limited proteolysis of 38 kDa CTGF primary translational product.

Two recombinant expression systems that produce these proteins were developed: one in mammalian cells and one in E. coli (Ball et al., Reproduction; 125: 271-84 2003; Ball et al., J Endocrinol; 176: R1-R7, 2003). Although the mammalian system was designed to produce 38 kDa CTGF, it also produces substantial amounts of lower mass CTGFs that are near-identical to those found in vivo. These low mass forms arise via proteolytic cleavage of the full-length protein (Ball et al., Reproduction; 125: 271-84, 2003). Briefly, 38 kDa hCTGF was cloned into pcDNA3.1 and transfected into CHO cells that were mutant for heparan sulfate and chondroitin sulfate and thus expected to liberate the recombinant CTGF protein into the medium rather than allowing it to become associated with cell-associated heparin-like molecules. One clone, termed DB1 was selected by limited dilution, and shown to secrete multiple CTGF isoforms. Serum-free conditioned medium was subjected to heparin-affinity and reverse-phase chromatography to isolate the individual CTGF proteins (Ball et al., Reproduction; 125: 271-84 2003).

Purification by reverse-phase HPLC demonstrated that each CTGF isoform supported adhesion of 3T3 cells to non-tissue culture plastic (Ball et al., Reproduction; 125: 271-84, 2003). Adhesion is a well-accepted and reproducible measure of CTGF activity. Structural analysis revealed the N-termini of each low mass protein as Ala 181 (20 kDa CTGF), Leu184 (18 kDa CTGF), Ala197 (16 kDa CTGF), or Gly253 (10 kDa CTGF) showing that this in vitro system produced the CTGF primary translational product which undergoes similar processing as found in the uterine tract. Using 4 liters of CHO conditioned medium approximately 0.5 mg, 1 mg, and 0.1 mg, respectively, of 38 kDa, 16-20 kDa, and 10 kDa CTGF (CTGF₁₋₄, CTGF₃₋₄ and CTGF₄, respectively) were produced. At 0.25-5 μg/ml each CTGF isoform demonstrated dose-dependent regulation of 3T3 cell adhesion which is consistent with previous reports for 38 kDa CTGF (Kireeva et al., Exp Cell Res; 233: 63-77, 1997). Each CTGF isoform stimulated 3T3 cell DNA synthesis, transdifferentiation of corneal epithelial cells into αSMA-expressing myofibroblasts and, when administered subcutaneously to neonatal mice, elicited the classic fibrogenic response that has previously been reported for 38 kDa CTGF or TGF-β (Hynes, Cell 110, 673-687, 2002), although 10 kDa CTGF actually gave the most robust response. These data show that the C-terminal ˜100 residues of CTGF (residues 247-349 of SEQ ID NO: 1) are sufficient to induce mitosis, adhesion, and differentiation in vitro or fibrosis in vivo (Ball et al., Reproduction; 125: 271-84, 2003; Brigstock et al., J Biol Chem; 272: 20275-82, 1997; Ball et al., Biol Reprod; 59: 828-35 1998).

To confirm the functionality of residues 247-349 of SEQ ID NO: 1 (containing module 4 alone; denoted as CTGF₄), this region of CTGF was produced as a maltose binding protein (MBP) fusion protein in E. coli as described in Ball et al., J Endocrinol; 176: R1-R7, 2003. After removal of MBP, C8 reverse-phase HPLC resulted in the separation of several CTGF proteins in the 8-10 kDa range, all of which promoted 3T3 cell adhesion. N-terminal sequence analysis of the most abundant 8 kDa and 10 kDa proteins demonstrated that they both commenced at Glu247 suggesting that the 8 kDa protein was C-terminally truncated (Ball et al., J Endocrinol; 176: R1-R7, 2003). HPLC-purified 10 kDa CTGF promoted 3T3 cell adhesion in a dose-dependent manner, with maximal binding at 8 ng/well. 10 kDa CTGF also promoted the dose-dependent adhesion of several additional cell types including vascular endothelial cells, intestinal epithelial, and hepatic stellate cells. The binding of each cell type to 10 kDa CTGF was completely inhibited by 5 μg/ml heparin. Also, the binding of all cell types to 10 kDa CTGF was reduced by EDTA treatment, though this effect was relatively modest in 3T3 cells. Thus, E coli-derived CTGF isoforms comprising essentially module 4 are intrinsically functional in the absence of the other constituent modules of CTGF.

### EXAMPLE 2

### Integrin-Mediated Adhesion of Activated HSC to CTGF

CTGF₄ is known to be expressed in hepatic stellate cells (HSC) and to induce proliferation and extracellular matrix (ECM) production in these cells. (Williams et al., J Hepatol 32, 754-761, 2000; Paradis et al., Lab Invest 82, 767-774, 2002). The following experiments were carried out to determine if HSCs adhere to CTGF. HSCs were isolated from normal Sprague-Dowley rate by sequential perfusion with pronase/collagenase and purified by density gradient separation as described in Vyas et al. (Gastroenterology 109: 889-898, 1995).

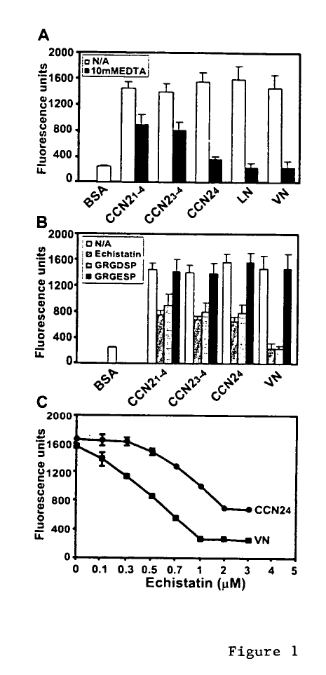

The cells were subsequently detached using 1 mM EDTA PBS (137 mM NaCl, 2.7 mM KCl, 4.3 mM Na₂HPO_{4,} 1.4 mM KH₂PO₄, pH 7.3), washed twice in DMEM and resuspended in serum free medium containing 0.5% BSA. The cells-were plated (1.25×10⁴ cells/well) in a 96 well plate coated with one of the following: 2 μg/ml CTGF isoform (CTGF₁₋₄, CTGF₃₋₄, CTGF₄), 1 μg/ml laminin, or 4 μg/ml vitronectin and incubated for 20 minutes at 37° C. Subsequently the cells were washed with PBS and the adherent cells were fixed with 10% formalin and stained with CyQUANT GR dye/cell lysis buffer (Molecular Probes , Eugene, Oreg.) according to the manufacturer's instructions. The number of cells that adhered to CTGF isoforms or ECM protein was quantified by measuring the sample fluorescence intensity at Ex/Em: 480/520 nm using a micro-plate reader (CytoFluor™ 2350). As shown in FIG. 1 (Panel A), the HSC cells adhered to each of the CTGF isoforms in a similar manner as they adhered to the ECM proteins vitronectin (VN) and laminin (LM).

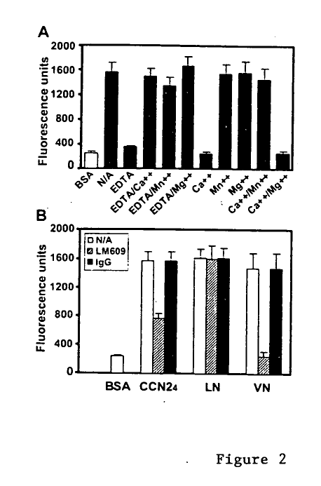

To determine if the CTGF-mediated HSC adhesion was carried out through integrin receptors, the experiment described above were carried out in the presence and absence of 10 mM EDTA. CTGF₁₋₄-, and CTGF₃₋₄-mediated HSC adhesion was decreased by 50% following by the addition of EDTA, whereas, CTGF₄-mediated HSC adhesion was inhibited by 90% (FIG. 1; panel A) in the presence of EDTA. Adherence was restored by the addition of 10 mM Mg²⁺ or Mn²⁺, while the presence of Ca²⁺ completely abolished cell adhesion to CTGF₄. Mn²⁺, but not Mg²⁺, was able to overcome the inhibitory effect (FIG. 2, Panel A). It is widely believed that Mn²⁺ induces conformational shifts that mimic the physiological activation of β₁ and β₃ integrins (Roberts et al., J Biol Chem 278: 1975-1985, 2003, van der Pauw et al., J Periodontal Res 37: 317-323, 2002) as Mn²⁺-induced activation leads to enhanced ligand-binding affinity and cell adhesion (Mould et al., J Biol Chem 277: 19800-19805, 2002, Loftus & Liddington J Clin Invest 100: S77-81, 1997, Lin et al., J Biol Chem 272: 14236-14243, 1197). The sensitivity of HSC adhesion to divalent cations suggests CTGF-adhesion is mediated through binging to an integrin being the adhesion receptor.

To further substantiate the role of integrin binding to CTGF, 6-residue RGD peptides and Echistatin peptide containing RGD were used to block the interaction of HSC with different CTGF isoforms. Synthetic peptides GRGDsp (1 mM), GRGEsp (1 mM), Echistatin (2 μM) or vehicle buffer alone were added to the, HSC culture medium 30 minutes prior to plating. As shown in FIG. 1 (Panel B), GRGDsp were able to block CTGF₁₋₄-, CTGF₃₋₄-, CTGF₄-mediated cell adhesion by 45%, 51% and 58%. Echistatin is a disintegrin with a binding preference for the β₃ integrin which is at least 500 times more effective than short RGDX peptides (Gould et al., Proc. Soc. Exp .Boil. Med. 195: 168-171, 1990). Echistatin inhibited the three CTGF isoforms-mediated cell adhesion by 58%, 62% and 69% respectively in a dose dependent manner. (FIG. 1; Panel C). Both RGD peptides resulted in a 100% decrease in HSC binding to vitronectin, which is a known ligand of αᵥβ₃. These experiments suggest that integrin αᵥβ₃ plays a role in CTGF₄-mediated cell adhesion.

### EXAMPLE 3

### CTGF₄-mediated Activated HSC Adhesion Partially Through Integrin αᵥβ₃

To further investigate the role of integrin binding in CTGF-mediated HSC adhesion, the expression αᵥβ₃ in HSC was analyzed by immunoprecipitation and western blotting. The α5 subunit was immunoprecipated with LM142, an anti-αᵥ antibody (Chemicon, Inc.). Subsequently, the immunoprecipitates were immunoblotted with LM609, an anti-β₃ antibody (Chemicon, Inc.). This analysis indicated that the 105 kDa protein corresponding to the β3 subunit of αᵥβ₃ was detected in the HSC cells confirming that integrin αᵥβ₃ was expressed in activated HSCs.

In addition, adhesion assays as described in Example 1 were carried out in CTGF₄ coated plates in the presence and absence of monoclonal antibody specific for αᵥβ₃ (LM609). As shown in FIG. 2 (Panel B), anti-αᵥβ₃ was able to inhibit adhesion of HSC to CTGF₄ by more than 60%, whereas mouse IgG had no inhibitory effect on CTGF₄-mediated cell adhesion. As expected, anti-α_{vβ}₃ completely blocked the cell adhesion to vitronectin, but no effect on laminin-mediated cell adhesion. Collectively, these results indicate that adhesion of HSC to CTGF₄ is partially mediated through integrin αᵥβ₃.

To further characterize the interaction between CTGF₄ and integrin αᵥβ₃, each CTGF isoforms (CTGF₁₋₄, CTGF₃₋₄, CTGF₄) was individually mixed with αᵥβ₃, followed by immunoprecipitation with rabbit anti-CTGF polyclonal antibody and immunoblotting with anti-αᵥβ₃. Human integrin αᵥβ₃ (2 μg) and each individual CTGF isoform (4 μg) were incubated in 1 ml NP40 buffer with rocking at 4° C. for 2 hours. As a control, 20 μM Echistatin was added prior to adding the CTGF isoforms. Subsequently, polyclonal rabbit anti-CTGF antibody (1:100) or mouse IgG was added to the complex and the mixtures were further incubated at 4° C. for 16 hours. After incubation, 25 ,μl of Protein A was added to each mixture for 1 hour. The samples were separated on 8% SDS-polyacrylamide gels, and transferred onto nitrocellulose. The membrane was then incubated with anti-human αᵥβ₃ monoclonal antibody (1:1000) diluted in TBS/Tween 20 (0.05%) containing 5% non-fat milk. Integrin αᵥβ₃ bound similarly to all CTGF isoforms. Echistatin fully competed for the binding of αᵥβ₃ with CTGF₄, indicating that Echistatin was able to directly and efficiently pre-occupy the αᵥβ₃ binding site.

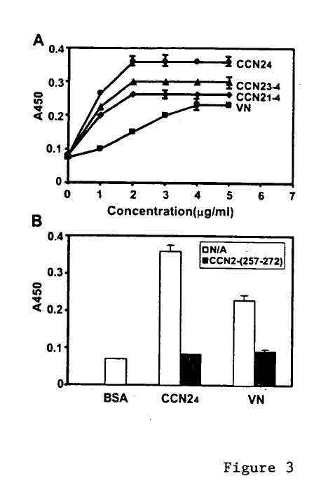

Solid phase binding studies were also performed to investigate the binding of CTGF isoforms and αᵥβ₃. Each CTGF isoform or vitronectin was individually coated onto microtiter wells at different concentrations, and the subsequent binding of αᵥβ₃ was detected by ELISA using an anti-human αᵥβ₃ monoclonal antibody. As shown in FIG. 3, (Panel A), all CTGF isoforms and vitronectin bound to αᵥβ₃ in a dose dependent manner. Saturation of binding occurred at 2 μg/ml for each CTGF isoform and 4 μg/ml for vitronectin. CTGF₄ had the strongest affinity for αᵥβ₃. These results from cell-free protein binding system are consistent with those obtained from HSC adhesion assays, and further substantiate that that module 4 of CTGF directly binds to αᵥβ₃.

### EXAMPLE 4

### Binding of Integrin αᵥβ₃ to CTGF[257-272]

In order to identify the αᵥβ₃ binding site in CTGF₄, eighteen synthetic peptides spanning the entire C-terminal region of CTGF₄ (residues 246-349 of SEQ ID NO: 1) were synthesized and used as potential blocking agents in the solid phase binding studies as described in Example 3. Microtiter wells (Dynex Technology) were pre-coated with different CTGF isoforms at desired concentrations at 4° C. for 16 hours, and then blocked with 2% BSA at room temperature for 2 hours. The plates were washed, four times with PBS, pH 7.3, containing 1 mM CaCl₂ and 1 mM MgCl₂. Integrin αᵥβ₃ (1 μg/ml) was pre-incubated with each synthetic peptide for 1 hour in blocking solution, and then added to each well, and incubated at room temperature for 3 hours. Integrin αᵥβ₃ was detected by successive incubation with anti-human αᵥβ₃ monoclonal antibody diluted in blocking solution (1:1000), and followed by HRP-conjugated goat anti-mouse IgG (1:4000). The color reaction was developed using the horseradish peroxidase ELISA reagents (Chemicon), and the absorbance at 450 nm was measured using Bio Assay Reader (HTS700).

As shown in FIG. 3 (Panel B), 35 μM peptide CTGF[257-272] SEQ ID NO: 2 completely inhibited αᵥβ₃ binding to CTGF₄, whereas, others CTGF₄ peptides : had no significant effect on binding. Peptide CTGF[259-272] (SEQ ID NO: 2), which covers 87.5% of the sequence of peptide CTGF[257-272] inhibited binding by 67%. Likewise, peptide CTGF[257-272] was capable of inhibiting αᵥβ₃ binding to vitronectin by 85%. This result suggest that CTGF[257-272] contains the CTGF-αᵥβ₃ binding site.

CTGF[257-272] promoted HSC adhesion in a concentration-dependent manner and with a saturation of 2 μg/ml, whereas CTGF[247-260] had no adhesion ability at 2 μg/ml. Other heparin-binding peptides demonstrated no or very weak adhesion ability at same concentration (2 μg/ml). CTGF[257-272]-mediated cell adhesion was abrogated by 2 μg/ml heparin or pre-incubation of the cells with heaprinase I. Echistatin and anti-αᵥβ₃ inhibited HSC adherence to CTGF[257-272] by 40% and 29% respectively. Taken together, these results demonstrate that CTGF[257-272] contains an αᵥβ₃ binding and the interaction of αᵥβ₃ with CTGF[257-272] is dependent on heparan sulfate proteoglycan on the cell surface.

### Example 5

### Adhesion of Activated HSC to CTGF Requires Cell Surface Heparin Sulphate proteoglycans (HSPG)

Previous studies showed that several regions in CTGF appeared to account for much of the heparin-binding ability of CTGF (Brigstock et al., J. Biol. Chem., 275: 24953-61, 1997). Experiments were carried out to investigate the role of heparan sulfate on the HSC surface in CTGF₄-mediated cell adhesion. Adhesion of HSC to CTGF₄ was completely abrogated by the presence of 2 μg/ml heparin in the plating medium, whereas heparin had little effect on vitronectin-mediated HSC adhesion. These results suggest that prior occupancy of the CTGF₄ heparin-binding sites by soluble heparin may interfere in its interaction with cell surface heparin sulphate proteoglycans (HSPGs), and thus inhibit HSC adhesion.

To further substantiate these results, HSC were treated with heparinase I, an enzyme that acts on highly sulfated heparan sulfate proteoglycans (Feitsma et al., J. Biol. Chem. 275: 9396-9402). Heparinase I treatment rendered the cells unable to adhere to CTGF₄, whereas the same treated cells adhered to vitronectin. Treatment of HSC with chondroitinase ABC had no effect on cell adhesion. These results indicate that cell surface heparan sulfates, but not chondroitin sulfates, contribute to HSC adhesion to CTGF₄.

To further characterize the role of HSPGs in, CTGF₄-mediated HSC adhesion, HSCs were cultured in the presence of sodium chlorate, an inhibitor of 3-phosphoadenosine 5 ′-phosphosulfate synthesis, to block sulfation of proteoglycans (Rapraegar et al., Science 252: 1705-1708, 1991). Inability of HSC to adhere to CTGF₄ in the presence of HSPGs was detected, whereas adhesion of the same cells to vitronectin was unaffected. The inhibitory effect of sodium chlorate on adhesion of HSCs to CTGF₄ was reversed by addition of 10 mM Na₂SO₄ to the culture medium, confirming that this inhibitory effect is mediated through a sulfation block. Taken together, these results suggest that cell surface HSPGs are necessary for adhesion of HSC to CTGF₄ and they may also function as an accessory molecule required for binding of CTGF₄ to integrin αᵥβ₃.

### EXAMPLE 6

### Site Directed Mutagenesis of Residues 257-272 of CTGF

Selection of Target Sequences

Modified alanine scanning using site-directed mutagenesis was used to mutate 4 amino acids at a time in the 16-residue region 257-272. Each of the four mutated proteins had 4 residues mutated to alanine:

TABLE 2

| | Residue | | |
|---|---|---|---|
| Mutant | Mutations | Sequence | SEQ ID NO: |
| M1 | I257A, R258A, | AAAAKISKPIKFELSG | 10 |
| | T259A, P260A | | |
| M2 | K261A, I262A, | IRTPAAAAPIKFELSG | 11 |
| | S263A, K264A; | | |
| M3 | P265A, I266A, | IRTPKISKAAAAELSG | 12 |
| | K267A, F268A | | |
| M4 | E269A, L270A, | IRTPKISKPIKFAAAA | 13 |
| | S271A, G272A | | |

The mutations were generated with site-directed mutagenesis using an adaptation of Kunkel's rapid oligonucleotide-directed procedure (Kunkel et al., Proc. Natl. Acad. Sci. U.S.A. 82: 488-92, 1985). Polymerase chain reaction (PCR) of human CTGF₄ was carried out using a Quickchange II (Stratagene, LA Jolla, Calif.) designed to introduced alanine substitutions. Briefly, 2 oligonucleotide primers at a time were simultaneously annealed to one strand of a denatured double-stranded CTGF₄ encoding template. The selection primer (5′-ATCGGGACATCTCCCGATCCCCTATG -3′, 57% GC; SEQ ID NO: 14) eliminates the unique Bgl II site in the pGEM-7zF(-) vector harboring CTGF[247-349], while the CTGF-mutation primers mutate the desired residues. The mutation primers are as follows: mutant #1: 5′-AGTGCGCCGCTGCTGCCAAAATCTCCAAGCCTATCAAGTTTGAGCTCTGGCTGCACC-3′ (SEQ ID NO: 15); mutant #2: 5′-GCATCCGTACTCCCGCGCGCCGCGCCTATCAAGTTTGAG-3′ (SEQ ID NO: 16); mutant #3: 5′-CCCAAAATCTCCAAGGCTGCCGCGGCTGAGCTTTCTGGC 3′ (; SEQ ID NO: 17); and mutant #4: 5′-GCCTATCAAGTTTGCGGCTGCTGCCTGCACCAGCATG-3′ (62% GC; SEQ ID, NO: 18) Elongation by T4 DNA polymerase resulted in the incorporation of both the selection (i.e. loss of Bgl II site) and CTGF mutations in the same strand. The DNA was then digested with Bgl II to linearize only the parent vector.

This DNA mixture was transformed into mismatch repair-deficient E. coli. The uncut, mutated DNA transformed more efficiently than the linear DNA with no mutations. After propagation of the plasmids in E. coli, the DNA was again digested with Bgl II to ensure that the non-mutated vector was eliminated. This mixture s was again transformed into mismatch repair-deficient E. coli and the mutant DNA was isolated. Two rounds of digestion and transformation ensured that a very high frequency of transformants carried the mutated plasmid. Mutants were expressed in E. coli as fusion proteins, cleaved, and purified as described in Ball et al., J Endocrinology, R1-R7, 2003.

Analysis of Mutant Proteins

The selected domain is not heparin-binding so the mutants were not expected to demonstrate altered affinity for heparin. Since heparin-affinity chromatography was used for purification, the heparin-binding properties of the mutant proteins were assessed and only those mutant peptides that exhibited heparin binding comparable to parental CTGF₄ were selected. Accordingly, all of the mutants exhibited comparable heparin binding properties to wild type CTGF₄. This was demonstrated by solid phase [³H]heparin binding. To verify that the tertiary structure of CTGF₄ was not radically altered by the mutations, immunoprecipitation with CTGF antisera confirmed that the protein was recognized and was efficiently immunoprecipitated.

The ability of mutant CTGF₄ to bind to integrin αᵥβ₃ was assessed by solid phase ELISA which measured the ability of purified integrin αᵥβ₃ to bind to the CTGF₄ mutants. Each mutant protein exhibited a dramatic loss of binding to αᵥβ₃ in the solid phase binding assay, showing that residues 257-272 of CTGF (SEQ ID NO: 1) contain critical determinants of integrin αᵥβ₃ binding. Mutant 2 was completely unable to support HSC adhesion, whereas all other mutants exhibited reduced levels of HSC adhesion that were, respectively, 33%, 80% and 46% those of wild-type CTGF₄. These data showed that although HSC adhesion involves the binding of residues 257-272 to integrin αᵥβ₃, this region of CTGF₄ likely contains additional determinants involved in HSC adhesion.

To analyze whether the, mutant CTGF₄ proteins such as those listed in table 1 and other mutant CTGF₄ proteins bind integrins such as integrin αᵥβ₃ or α₅β₁, assays such as those described above may be carried out using cells known to express the integrin of interest, such HSCs, PSCs and HUVECs. If cell adhesion is lessened but still measurable in the mutant proteins, Western blotting is used to assess phospho-FAK and phospho-MAPK in lysates from the adherent cells, the levels of which are expected to be severely compromised as compared to parental CTGF₄.

Once the effect of these mutations is established, site directed mutagenesis is again used with point mutations to specifically map the important residues. Alanine, as well as conservative substitutions are used for this specific mapping. In this manner, it is possible to identify the critical residues and to create a mutant form of CTGF₄ in which all such residues have been altered. These data are then translated to the CTGF₁₋₄ protein in which the same mutations are made. This will verify that the lack of integin αᵥβ₃ binding or cell adhesion holds true in the context of the full-length protein. Finally, the effect of the mutations on biological readouts other than adhesion is assessed, including proliferation, migration, TIMP production, angiogenesis, and fibrosis.

### EXAMPLE 7

### Development of Short Peptide Antagonist Between CTGF and an Integrin

Functionally, the ability of CTGF[257-272] to inhibit integrin αᵥβ₃-CTGF₄ interactions is comparable to the inhibition by RGD peptides. Therefore, it is contemplated that shorter peptides derived from within residues 257-272 will retain antagonist properties and will serve to map the key residues involved. It is possible that non-adjacent residues are also involved in integrin αᵥβ₃ or other integrin binding. Overlapping peptides, each containing 5 residues, that span the entire active domain are synthesized. To adequately investigate those residues at the start and end of the sequence, the analysis includes peptides that are N-terminal and C-terminal to these regions. The peptides to be made will correspond to residues 252-256, 253-257, 254-258, 255-259, 256-260, 257-261, 258-262, 259-263, 260-264, 261-265, 262-266, 263-267, 264-268, 265-269, 266-270, 267-271, 268-272, 269-273, 270-274, 271-275, 272-276, and 273-277 of SEQ ID NO: 1.

Peptides are tested for their ability to inhibit HSC or HUEC adhesion as well as to inhibit direct binding of integrin αᵥβ₃ or other integrins to CTGF₄ in a solid phase ELISA as described in Example 3. For example, if three adjacent residues are involved, it is expected that three of the peptides will be active as they will each contain the triplet sequence in question (for example, residues 262-264 are in peptides 260-264, 261-265, and 262-266). Once the peptides are screened in this way, further analysis is carried out to identify those that are shown to be active by testing them on CTGF₁₋₄-coated plates in both cell adhesion assays and ELISA as described in Example 2. Once the residues are identified, scrambled peptides of the same composition but different sequence will be generated to verify that the absolute sequence is a prerequisite of activity.

### EXAMPLE 8

### Development of Peptide Antibodies that Block CTGF-Integrin αᵥβ₃ Binding

CTGF peptides such as CTGF[257-272], CTGF[285-291] and other peptides of the invention are used to create antibodies that may be used to block the binding of CTGF₄ to an integrin such as αᵥβ₃ or α₅β₁. Rabbit antisera are produced against a CTGF-MAP peptide, affinity-purified, and tested. for dose-dependent inhibition of integrin αᵥβ₃ binding to CTGF₁₋₄ or CTGF₄ in a solid phase ELISA. It is expected that the antibody will inhibit the binding of the integrin to both CTGF isoforms. Preimmune IgG will serve as a negative control. To assess the effect of the antibody on CTGF-mediated cell adhesion, cells are plated onto CTGF₁₋₄- or CTGF4-coated wells that have been pre-incubated with anti-CTGF peptide. The level of HSC or HUVEC binding is expected to be reduced though may not be totally inhibited given that HSPGs are also involved in CTGF-mediated cell adhesion.

### EXAMPLE 9

### Adhesion of PSC to CTGF is Mediated via Integrin α₅β₁ and HSPG

Pancreatic stellate cells (PSC) are found in peri-acinar and peri-ductular locations and contain vitamin A in cytoplasmic lipid droplets in the normal pancreas. When cultured in vitro, PSC autonomously differentiate into myofibroblast-like cells that express α-smooth muscle actin (α-SMA) and produce collagen types I and III, laminin and fibronectin (FN). It is postulated that the in vivo corollary of this “activation” occurs during pancreatic injury in response to various stimuli (e.g., growth factors, proinflammatory cytokines, oxidant stress) causing PSCs to transform into highly fibrogenic cells that produce large amounts of extracellular matrix-containing fibrillar collagen (Apte et al., Gut 44:534-41, 1999, Mews et al., Gut 50:535-41, 2002, Phillips et al., Gut 52:275-82, 2003, Apte et al., Pancreas 27:316-20, 2003). The acquisition of an activated pro-fibrotic phenotype by PSC is akin to a similar process in hepatic stellate cells (HSC) which is a pivotal event during fibrosing liver injury. Thus, it was of interest to determine if PSC express CTGF.

Rat PSCs were isolated by a modification of the method described by Phillips et al., Gut; 52:677-82, 2003. Briefly, pancreatic tissue was digested in situ with 0.03% collagenase P (Roche, Indianapolis, Ind.) in HBSS by perfusion through the thoracic aorta. This protocol was approved by the Institutional Animal Care and Use Committee of Children's Research Institute, Columbus, Ohio. The tissue was pooled, minced with scissors, and then digested with 0.05% collagenase P, 0.02% protease XIV (Sigma, St. Louis), and DNase I (Roche, Indianapolis, Ind.) in HBSS. The resulting cell suspension was centrifuged in a 12% Optiprep gradient at 1400 g for 14 minutes. Stellate cells separated into a hazy band just above the interface of a the gradient and the aqueous buffer. This band was harvested, and the cells washed and resuspended in DMEM supplemented with 25 mM Hepes buffer, 10% FBS, and 100 U/ml penicillin in, 100 μg/ml streptomycin. The, cells were maintained at 37° C. in a humidified atmosphere of 5% CO₂/95% air.

Production of CTGF by PSCs was confirmed by immunoprecipitation, which revealed the presence of the CTGF₁₋₄ protein (38 kDa) in both cell lysates and conditioned medium using the method described in Ball et al. (Reproduction 2003;125:271-84). CTGF₁₋₄ levels, as wells as CTGF₃₋₄ and CTGF₄ levels, were significantly increased (P<0.01) in both compartments following treatment of the cells with TGF-β1 (20 ng/ml; Life Technologies, Grand Island, N.Y.). TGF-β1 is a well characterized transcriptional activator of CTGF in other cell types. To further examine the mechanism by which TGF-β1 and other fibrogenic stimulators could influence CTGF production in PSCs, CTGF promoter activity in PSCs was evaluated using the luciferase reporter, pCCN2-Luc, as described in Gao et al., (J Hepatol 2004;40:431-8). The activity of pCCN2-Luc in PSC was significantly increased following stimulation by TGF-β1, PDGF, ethanol or acetaldehyde (P<0.01 compared with no stimulation) with TGF-β1 exhibiting the strongest activation of pCCN2-Luc (P<0.05 compared among four stimulators).

As assessed immunohistochemically, activated PSCs were positive for production of vimentin, desmin, and α-SMA. Incubations with mouse monoclonal antibodies to α-SMA (1:40; Dako, Glostrup, Denmark), vimentin (1:30; Dako, Glostrup, Denmark), or desmin (1:20; Sigma, St. Louis, Mo.) were performed at room temperature for 1 hour, followed by FITC- or TRITC-labeled goat anti-mouse antibodies (Sigma, St. Louis, Mo.) for 30 minutes. All incubation steps were followed by three washes with PBS for 5 minutes. Slides were mounted with glycergel mounting medium. Controls consisted of omission of the primary antibodies. For CTGF staining, slides were successively incubated with anti-CCN2(81-94) peptide antibody (1:100) (as described in Steffen et al., Growth Factors 1998;15:199-213), biotinylated anti-IgG and streptavidin-HRP. Development of the chromogenic color reaction was accomplished using DAB substrate. All of the cells were positive for cytoplasmic CTGF.

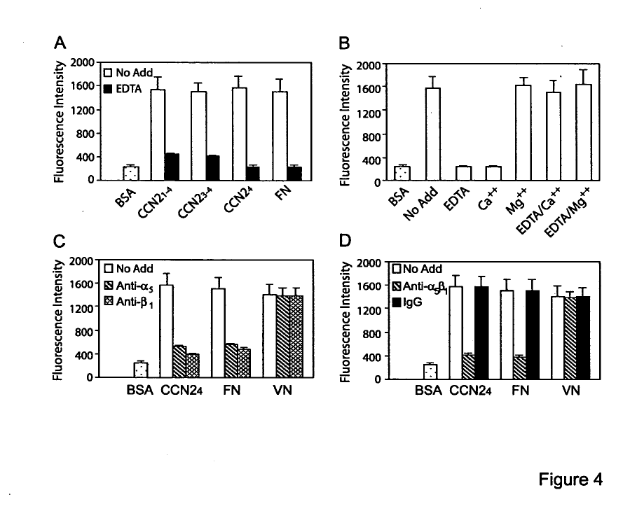

The ability of activated PSC to adhere to non-tissue culture plastic microtiter wells coated with recombinant CTGF₁₋₄, CTGF₃₋₄ or CTGF₄ was investigated. The solid phase binding assay was carried out as described in Example 2. Wells coated with 2 μg/ml CTGF (CTGF₁₋₄, CTGF₃₋₄ or CTGF₄) significantly promoted PSC adhesion (P<0.01 vs. control) and supported a level of binding that was comparable to wells coated with 2 μg/ml FN, as displayed in FIG. 4 (Panel A). To assess the possibility that CTGF-mediated PSC adhesion involved cell surface integrins, the effect of divalent cations on cell adhesion was examined, as described in Example 2. Following incubation of the cells with 5 mM EDTA, CTGF-mediated (CTGF₁₋₄, CTGF₃₋₄ or CTGF₄) PSC adhesion was significantly decreased (P<0.01 vs. untreated cells), but was restored by addition of the divalent cations Ca²⁺ (10 mM) or Mg²⁺ (10 mM) as displayed in FIG. 4 (Panel B).

Since the cation dependence of PSC adhesion was consistent with the possible involvement of integrins in this process, the effect of pre-treatment of the cells with specific antibodies against individual integrin subunits prior to exposure to CTGF₁₋₄ was investigated. Of the antibodies tested, only those raised against the α₅ or β₁ subunits (25 μg/ml) were effective in blocking PSC adhesion to CTGF₁₋₄ and CTGF₄. Moreover, PSC adhesion to CTGF₁₋₄ or CTGF₄ was inhibited by anti-α₅β₁; this antibody was also capable of blocking FN-mediated adhesion, as expected, since FN is a principal ligand for integrin α₅β₁, but not VN-mediated adhesion A representative data set for CTGF₄ adhesion is depicted in FIG. 4 (Panels C and D). Collectively these data indicated that PSC adhesion to CTGF is dependent on integrin α₅β₁. To verify production of integrin α₅β₁ by PSC, activated cells underwent sequential anti-integrin β₁ immunoprecipitation followed by an anti-integrin α₅ immunoblot. A 145-kDa immunoreactive protein was detected on the Western blot, which corresponded to the predicted size of the integrin as subunit and thus confirmed that integrin α₅β₁ was produced by activated rat PSCs used in the disclosed studies.

Because CTGF is a heparin-binding protein, this property was further investigated. Adhesion of PSC to CTGF₁₋₄ and CTGF₄ were blocked by the presence of 2 μg/ml heparin in the plating medium, whereas the same concentration of heparin had little effect on adhesion of PSCs to FN. These results suggested that soluble heparin occupancy of the heparin-binding sites in CTGF might cause interference with the ability of CTGF to bind to its cell surface adhesion receptors. To confirm this observation, cell surface HSPGs were either removed from PSCs by treatment of the cells with heparainase I (an enzyme that acts on highly sulfated HSPGs, Fietsma et al., J Biol Chem 2000;275:9396-402) or sulfation was blocked by pre-incubation of the cells with sodium chlorate (Rapraeger, et al., Science 1991;252:1705-8). Each treatment significantly reduced the ability of PSCs to adhere to CTGF₁₋₄ and CTGF₄, yet did not affect their adhesion to FN. The inhibitory effect of sodium chlorate on the adhesion of PSCs to CTGF₁₋₄ and CTGF₄ was reversed by addition of 10 mM Na₂SO₄ to the culture medium, confirming that this inhibitory effect was mediated through a sulfation block (Rapraeger, et al., Science 252:1705-8, 1991). Taken together, these results suggest that cell surface HSPGs cooperate with CTGF in supporting cell adhesion via integrin α₅β₁.

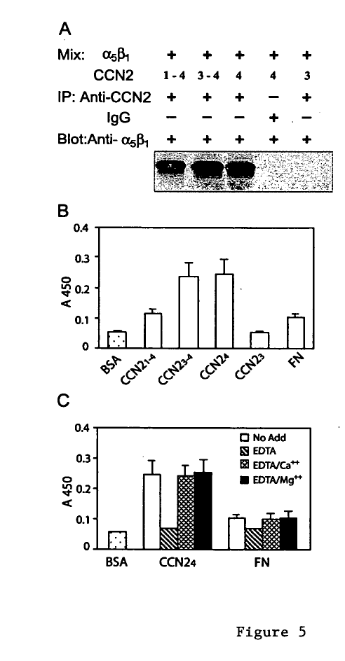

In addition, cell-free binding assays were developed to verify that CTGF is a ligand of integrin α₅β₁. In one approach, CTGF (4 μg/ml CTGF₁₋₄, CTGF₃₋₄, CTGF₄ or CTGF₃) was incubated in 1 ml NP40 buffer solution with purified integrin α₅β₁ prior to sequential immunoprecipitation with rabbit anti-CTGF polyclonal antibody (as described in Ball et al., Reproduction 125: 271-284, 2003) and immunoblotting with anti-integrin α₅β₁ (Chemicon, Inc., Temicula, Calif.). As shown in FIG. 5 (Panel A), a direct binding between integrin α₅β₁ and CTGF protein in solution was demonstrated, as revealed by the detection of integrin α₅β₁ in reactions containing anti-CTGF IgG but not normal IgG.

In a second approach, a solid phase binding assay was used in which CTGF₁₋₄ or FN were individually coated onto microtiter wells that were subsequently incubated with purified integrin α₅β₁. The presence of immobilized integrin α₅β₁ was detected by ELISA using an anti-integrin α₅β₁ antibody similar to the methods described in Example 3 above. In addition, CTGF₁₋₄ bound to integrin α₅β₁ in a dose-dependent manner, with maximal binding at 2 μg/ml CTGF. This level of CTGF₁₋₄ (2 μg/ml) binding was the same as that elicited by 2 μg/ml FN, a well known ligand of integrin α₅β₁. In a similar experiment, 2 μg/ml of CTGF₁₋₄;CTGF₃₋₄ or CTGF₄ were individually precoated onto microtiter wells. Using the solid-phase assay, integrin α₅β₁ bound to CTGF₁₋₄, CTGF₃₋₄ or CTGF₄ See FIG. 5 (Panel B). The binding of integrin α₅β₁ to CTGF4 was blocked by the addition of EDTA and this inhibition was reversed by the addition of cations Ca²⁺ and Mg²⁺ Collectively, results from both types of cell-free binding assay approaches clearly indicated that CTGF binds directly to integrin α₅β₁.

### EXAMPLE 10

### Stimulation of PSC Migration, Proliferation and Collagen Synthesis by α₅β₁

To examine whether CTGF plays a role in PSC migration, a chemotaxis assay was carried out in which the migratory behavior of PSCs in the upper chamber of a culture insert was assessed following addition of CTGF to the lower chamber. CTGF (CTGF₁₋₄, CTGF₃₋₄ or CTGF₄) induced PSC migration across the polyethylene membrane in the culture insert. PSC migration was also promoted by FN, PDGF or TGF-β1. These results are consistent with the cell adhesion data described in Example 8. PSC migration in response to either FN, CTGF₁₋₄ or CTGF₄ was blocked by anti-integrin α₅β₁ antibody, while the effect of CTGF, but not that of FN, was blocked by the presence of heparin. These data indicate that CTGF-stimulated cell migration involves interactions of CTGF with both integrin α₅β₁ and heparin-like molecules.

To measure the effect of CTGF on cell proliferation, DNA synthesis was measured by ³H incorporation in PSCs. The cells were incubated for 24 hours with 0.2 μCi [³H] thymidine in each well (24-well plate). The incorporation of [³H] thymidine into PSC DNA was determined using a scintillation counter after the cells had been washed with PBS, fixed with 10% methanol, treated with cold 5% trichloroacetic acid, and lysed in 0.3 N NaOH. Direct stimulation of activated rat PSC with either CTGF₁₋₄ or PDGF significantly enhanced cell proliferation, whereas stimulation with FN or TGF-β1 did not induce proliferation. This effect of CTGF₁₋₄ was observed when it was tested in the assay in the presence of 0.1% serum but not in serum-free medium, indicating that its effects on;DNA synthesis requires one or more serum components.

CTGF₁₋₄ also. significantly increased collagen I mRNA expression in PSCs indicating that CTGF contributes to the pro-fibrogenic phenotype of PSCs by promoting synthesis of fibrillar collagen.

### EXAMPLE 11

### Binding of Integrin α₅β₁ to CTGF[285-291]

In order to identify the α₅β₁ binding site in CTGF₄, eleven synthetic peptides spanning the entire C-terminal region of CTGF₄ (residues 247-349 of SEQ ID NO: 1) were synthesized and are set out below in Table 3. These peptides were used as potential ligands for PSCs in a cell adhesion assay.

TABLE 3

| | | Residues of |
|---|---|---|
| Peptide | Sequence | SEQ ID NO:1 |
| P1 | EENIKKGKKCIRTP | 247-260 |
| | (SEQ ID NO: 26) | |
| P2 | IRTPKISKPIKFELSG | 257-272 |
| | (SEQ ID NO: 2) | |
| P3 | TPKISKPIKFELSGCTS | 259-275 |
| | (SEQ ID NO. 27) | |
| P4 | TSMKTYRAKF | 274-286 |
| | (SEQ ID NO: 28) | |
| P5 | GVCTDGR | 285-291 |
| | (SEQ ID NO: 9) | |
| P6 | CTPHRTTTLPVEFK | 293-306 |
| | (SEQ ID NO: 29) | |
| P7 | FKCPDGEVMKKNMMFIKT | 305-322 |
| | (SEQ ID NO: 30) | |
| P8 | MFIKTCA | 318-324 |
| | (SEQ ID NO: 31) | |
| P9 | ACHYN | 324-328 |
| | (SEQ ID NO: 32) | |
| P10 | CPGDNDIFESLY | 329-340 |
| | (SEQ ID NO: 33) | |
| P11 | LYYRKMYGDMA | 339-349 |
| | (SEQ ID NO: 34) | |

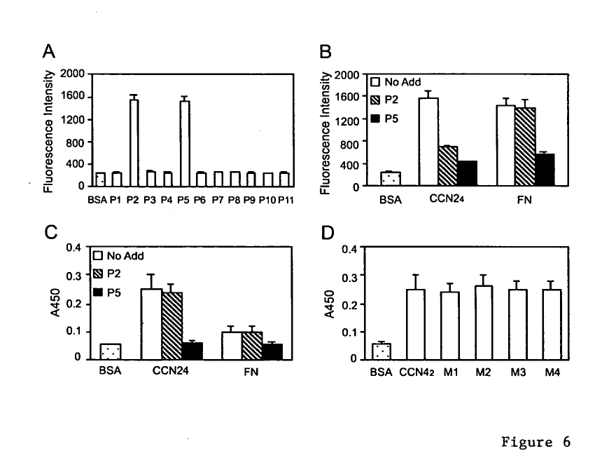

To identify the peptides that bind to PSCs, cell adhesion assays were carried out as described in Example 3. For this assay, 96-well plates were coated with 2 μg/ml of the synthetic peptides spanning the 103 C-terminal residues of CTGF as set out in Table 2 (peptides P1-P11). As shown in FIG. 6 (Panel A), peptide P2 (CTGF[257-272]; SEQ ID NO: 2) and peptide P5 (CTGF[285-291]; SEQ ID NO: 9) were able to support the adhesion of PSC.

To further investigate the peptide binding, P2 or P5 (10 μM) or vehicle buffer alone (no add) were added to PSC cell suspensions for 30 minutes at room temperature. The cells were then plated on microtiter wells that had been precoated with CTGF₄ (2 μg/ml) or FN (4 μg/ml), as described in Example 3. As shown in FIG. 6 (Panel B), 35 μM of P2 peptide (CTGF[257-272]) or P5 peptide (CTGF [285-291]) completely inhibited PSC binding to CTGF₄. These data show that P2 and P5 contain critical domains for PSC adhesion. Moreover, peptide P5 (CTGF [285-291]) was able to block FN-mediated PSC adhesion, which is dependent on integrin α₅β₁.

To directly analyze whether α₅β₁ integrin binds to CTGF₄, microtiter wells coated with CTGF₄ (2 μg/ml) or FN (4 μg/ml) were used in an ELISA assay to detect integrin binding, as described in Example 3. For these assays, 1 μg/ml integrin α₅β₁, alone or in the presence of 35 μM of P2peptide (CTGF[257-272]) and 35 μM of P5 peptide (CTGF [285-291]), were added to the precoated plates. CTGF₄ bound strongly to integrin α₅β₁ (FIG. 6, Panel C), thus verifying that CTGF₄ is a ligand for integrin α₅≈₁. The binding between CTGF₄ and integrin α₅ β₁ was blocked by peptide P5 (CTGF [285-291]) or peptide P2 (CTGF[257-272]). P5 also blocked the cell-free binding of integrin α₅β₁ to FN, whereas P2 did not block α₅β₁ binding to FN.

Since peptide P2 promoted PSC adhesion, the role of this domain in binding to integrin α₅β₁ was further investigated. This was accomplished by direct binding analysis in which mutant proteins harboring mutations in the P2 region were tested for their ability to bind to integrin α₅β₁ Microtiter wells were coated individually with CCN2₄ (CCN2₄-MBP, 8 μg/ml), or 8 μg/ml of one of the four mutant peptides described in Example 6 (i.e., M1-SEQ ID NO: 10, M2-SEQ ID NO: 11, M3-SEQ ID NO: 12, M4-SEQ ID NO: 13) at 4° C. for 16 hours. These precoated plates were used in an ELISA assay to detect integrin α₅β₁ (1 μg/ml) binding, as described in Example 3. The binding of CTGF₄ to integrin α₅β₁ was not reduced in the presence of any of the mutant proteins that the P2 site does not play a critical. role in the binding of CTGF₄ to integrin α₅β₁. This conclusion is supported by the observation that P2 peptides did not affect the ability of CTGF₄ to bind to integrin α₅α₁ (FIG. 6, Panel C). The fact that P2 peptide can promote cell adhesion (FIG. 6, Panel A) or inhibit CTGF4-mediated cell adhesion (FIG. 6, Panel B) indicates the presence of a cell binding determinant in the P2 sequence that interacts with an undetermined moiety but one that is not integrin α₅β₁. This is supported by the finding the adhesion to FN, which is dependent on integrin α₅β₁ was unaffected by P2 (FIG. 6, Panel B).

On the other hand, P5 peptide directly promoted cell adhesion (FIG. 6, Panel A), inhibited either CTGF₄- or FN-mediated cell adhesion (FIG. 6, Panel B), and blocked the direct binding between integrin α₅β₁ and either CTGF₄ or FN. These data show that P5 contains a binding determinant of integrin α₅β₁. Collectively, these results demonstrate that CTGF binds to integrin α₅β₁ through the binding site sequence of GVCTDGR (CTGF [285-291]).

1. An isolated peptide comprising a fragment of the amino acid sequence of SEQ ID NO: 1 wherein the peptide binds to an integrin.

2. The peptide of claim 1 wherein the peptide binds to integrin αᵥβ₃.

3. The peptide of claim 1 wherein the peptide binds to integrin α₅β₁.

4. The peptide of claim 1 wherein the peptide sequence comprises residues 257-272 of SEQ ID NO: 1.

5. The peptide of claim 1 wherein the peptide sequence comprises residues 285-291 of SEQ ID NO: 1.

6. The peptide of claim 1, wherein the peptide inhibits the binding of connective tissue growth factor (CTGF) to an integrin.

7. The peptide of claim 6 wherein the integrin is αᵥβ₃.

8. The peptide of claim 6 wherein the integrin is α₅β₁.

9. An isolated peptide comprising the amino acid sequence of SEQ ID NO: 2.

10. An isolated peptide comprising the amino acid sequence of SEQ ID NO: 19.

11. An isolated polynucleotide encoding the peptide according to any one of claims 1-10.

12. A method of inhibiting the binding of connective tissue growth factor (CTGF) to an integrin comprising administering an effective amount of the peptide of claim 6.

13. The peptide of claim 6, wherein the peptide inhibits a connective tissue growth factor (CTGF) biological activity selected from the group consisting of extracellular matrix production, cell proliferation, cell migration, cell cycle progression, cell differentiation, cell adhesion and chemotaxis.

14. A method of inhibiting a connective tissue growth factor (CTGF) biological activity comprising administering an effective amount of the peptide of claim 13.

15. The peptide of claim 1, wherein the peptide stimulates the binding of connective tissue growth factor (CTGF) to an integrin.

16. The peptide of claim 15 wherein the integrin is αᵥβ₃.

17. The peptide of claim 15 wherein the integrin α₅β₁.

18. A method of stimulating the binding of connective tissue growth factor (CTGF) to an integrin comprising administering an effective amount of the peptide of claim 15.

19. A peptide of claim 15, wherein the peptide stimulates a connective tissue growth factor (CTGF) biological activity selected from the group consisting of extracellular matrix production, cell proliferation, cell migration, cell cycle progression, cell differentiation, cell adhesion and chemotaxis.

20. A method of stimulating a connective tissue growth factor (CTGF) biological activity comprising administering an effective amount of the peptide of claim 19.

21. An antibody that specifically binds to the peptide of claim 1.

22. An antibody that specifically binds to the peptide of claim 9.

23. An antibody that specifically binds to the peptide of claim 10.

24. The antibody according to any one of claims 21-23, wherein the antibody inhibits the binding of connective tissue growth factor (CTGF) to an integrin.

25. The antibody of claim 24, wherein the integrin is αᵥβ₃ or α₅β₁.

26. A method of inhibiting binding of connective tissue growth factor (CTGF) to an integrin comprising administering the antibody of claim 24.

27. The antibody according to any one of claims 21-23, wherein the antibody inhibits a connective tissue growth factor (CTGF) biological activity selected from the group consisting of: extracellular matrix production, cell proliferation, cell migration, cell cycle progression, cell differentiation, cell adhesion and chemotaxis.

28. A method of inhibiting connective tissue growth factor (CTGF) biological activity comprising administering an effective amount of the peptide of claim 27.

29. A method of stimulating wound healing comprising administering an effective amount of the peptide according to any one of claims 15-17 to a mammal in need.

30. A kit for stimulating wound healing in a mammal in need, wherein the kit comprises a peptide according to any one of claims 15-17 and a set of instructions for administering the peptide.

31. A method of treating a connective tissue growth factor (CTGF)-related disorder comprising administering an effective amount of the peptide of any one of claims 6-8 and 13 to a mammal in need.

32. A kit useful for treating a connective tissue growth factor (CTGF)-related disorder in a mammal in need, wherein the kit comprises a peptide according to any one of claims 6-8 and 13 and a set of instruction for administering the peptide.
